(19) United States
(12) Patent Application Publication
(10) Pub. No.: US 2019/0105340 A1
(43) Pub. Date: Apr. 11, 2019

(54) METHODS AND COMPOSITIONS FOR TARGETING VASCULAR MIMICRY

(71) Applicant: New York Genome Center, Inc., New York, NY (US)

(72) Inventors: Greg Hannon, Longstowe (GB); Ian Cannell, New York, NY (US)

(21) Appl. No.: 16/152,080

(22) Filed: Oct. 4, 2018

Related U.S. Application Data
(60) Provisional application No. 62/568,672, filed on Oct. 5, 2017.

(51) Int. Cl. A61K 31/7072; A61P 35/00; A61P 35/04; A61K 39/395; A61K 31/404

(52) U.S. Cl. CPC A61K 31/7072; A61P 35/00; A61K 31/404; A61K 39/3955; A61P 35/04

(57) ABSTRACT

A method for increasing the sensitivity of a tumor to anti-angiogenic therapy comprises treating a patient having a tumor with an anti-angiogenic therapeutic composition or compound and substantially simultaneously inhibiting vascular, or vasculogenic, mimicry (VM). In one embodiment, such a method includes the inhibition of VM by administering a therapeutic compound that inhibits the activity or pathway of the transcription factor FOXC2. In another embodiment, such a method includes administering a therapeutic compound that activates or enhances the activity or pathway of IRE1 and/or inhibits its target genes. In another embodiment, all three compositions are administered to the subject either simultaneously or sequentially.

### CROSS-REFERENCE TO RELATED APPLICATIONS

This application claims the benefit of the priority of U.S. Provisional Patent Application No. 62/568,672, which application is incorporated herein by reference.

### STATEMENT REGARDING FEDERALLY SPONSORED RESEARCH OR DEVELOPMENT

This invention was made with government support under Grant Nos. 5R37GM062534-17, awarded by the National Institutes of Health. The government has certain rights in this invention.

### BACKGROUND OF THE INVENTION

Rapidly dividing tumors require a plethora of nutrients and other factors supplied by the blood stream to thrive and spread. Therefore, many tumors release factors that stimulate the growth of normal endothelial cells¹ (i.e., cells that line blood vessels) in the host through a process called angiogenesis. Over the past decades^{1,2}, it was proposed that inhibition of angiogenesis would likely starve the tumor of essential nutrients and oxygen leading to tumor cell death. Thus, many pharmaceutical companies developed drugs targeting critical mediators of angiogenesis, such as the vascular endothelial growth factor (VEGF) family of growth factors, to much excitement among the oncology community. See, e.g., Liang³⁹ and references cited therein. However, clinical trials of anti-angiogenic agents, such as Avastin®/bevacizumab (Genentech), have largely been disappointing with most patients showing transient responses followed by inevitable resistance.³ This is especially pertinent in metastatic breast cancer where, after initial approval, the FDA has since rescinded its approval of Avastin®/bevacizumab for use in breast cancer.

Since the emergence of the original theory behind targeting critical mediators of angiogenesis to treat cancer, it has been determined that heterogeneous tumors can employ alternative mechanisms of vascularization⁴. One such mechanism relies on vasculature formed by tumor cells themselves that differentiate into endothelial-like cells to form extra-cellular matrix (ECM)-rich tubular structures that are essentially pseudo blood vessels, a process termed vascular mimicry or vasculogenic mimicry (VM). VM was first described in the early 1990s⁵ and has since been seen in a broad spectrum of tumor types, where its presence is almost universally a poor prognostic indicator⁶. Subpopulations of tumor cells that can form VM channels endow tumors with an alternative vascular system for nutrient supply without requiring host vessel growth through angiogenesis⁴³ and, as such, have been postulated to underlie poor responses to anti-angiogenic agents. However, an understanding of how tumor cells acquire VM capabilities and whether VM underlies failure of anti-angiogenic therapy, as well as how to use this information enable the development of effective therapeutic interventions for cancer therapy are lacking and no anti-VM therapies exist due to a poor understanding of the details of how VM occurs.

### SUMMARY OF THE INVENTION

Through genetic barcoding of individual breast cancer cells, we discovered a critical role for VM in promoting metastasis by facilitating tumor cell entry into the blood stream⁷. This analysis uncovered two novel regulators of VM (SerpinE2 and SLPI), and provided a comparative system through which to understand the underlying biology of VM. Utilizing this comparative system, we have now identified additional critical pathways controlling the establishment and maintenance of VM namely, the Forkhead box protein C2 (FOXC2; also known as MFH1) and inositol-requiring enzyme 1 (IRE1) pathways, respectively.

In one aspect, a method for increasing the sensitivity of a tumor to anti-angiogenic therapy comprises treating a patient having a tumor with an anti-angiogenic therapeutic composition or compound and substantially simultaneously inhibiting vascular, or vasculogenic, mimicry (VM). In one embodiment, such a method includes the inhibition of VM by administering a therapeutic compound that inhibits the activity or pathway of the transcription factor FOXC2 or inhibits a FOXC2 pathway target. In another embodiment, such a method includes administering a therapeutic compound that activates or enhances the activity or pathway of IRE1 or inhibits/diminishes the activity of its target genes.

In another aspect, a method for increasing the sensitivity of a tumor to anti-angiogenic therapy comprises treating a patient having a tumor with an anti-angiogenic therapeutic composition or compound, and a therapeutic compound that inhibits the activity or pathway of the transcription factor FOXC2, and a therapeutic compound that activates or enhances the activity or pathway of IRE1 or inhibits/diminishes the activity of its target genes.

In still a further aspect, a therapeutic composition for inhibiting tumor vascularization and vasculogenic mimicry comprises in a suitable pharmaceutical carrier, an anti-angiogenic therapeutic compound and at least one or a combination of (a) a therapeutic compound that inhibits the activity or pathway of the transcription factor FOXC2; and (b) a therapeutic compound that activates or enhances the activity or pathway of IRE1 or inhibits/diminishes the activity of its target genes.

In a further aspect, a therapeutic regimen comprises (a) administering to a subject with a tumor an anti-angiogenic therapeutic composition or compound; and (b) administering to said subject substantially simultaneously or sequentially, at least one of i) a therapeutic compound that inhibits the activity or pathway of the transcription factor FOXC2, and ii) a therapeutic compound that activates or enhances the activity or pathway of IRE1 or inhibits/diminishes the activity of its target genes.

In still another aspect, a composition or reagent for diagnosing the existence or evaluating the progression of cancer in a mammalian subject are provided, which comprise multiple polynucleotides or oligonucleotides. Each polynucleotide or oligonucleotide hybridizes to a different gene, gene fragment, gene transcript or expression product in a sample selected from gene targets that experience changes in expression during vascular mimicry.

Yet another aspect involves a method for diagnosing the existence or evaluating the progression of a cancer in a mammalian subject comprising identifying changes in the expression of multiple genes in the sample of said subject, said genes selected from genes that change expression in response to increasing or decreasing vascular mimicry. Such methods may be used to assess the efficacy of the treatment methods also described herein.

Other aspects and advantages of these compositions and methods are described further in the following detailed description of the preferred embodiments thereof

### BRIEF DESCRIPTION OF THE DRAWINGS

FIGS. 1A-1G demonstrates that a mouse model of breast cancer heterogeneity implicates vascular mimicry (VM) as a driver of metastasis.

FIG. 1A is a schematic of a mouse model of breast cancer heterogeneity, including single cell clone barcoding and isolation from 4T1 parental cells.

FIG. 1B is a gel showing relative distribution of individual barcoded clones following mammary fat-pad transplantation of the 23 pooled single cell clones.

FIG. 1C is a dot graph showing an RNA-Seq analysis of all 23 single cell clones. Shown on the X axis are a subset of genes that were significantly up-regulated in clones E and T relative to all other clones.

FIG. 1D is a bar graph showing the effect of SerpinE2 or SLPI knockdown on lung metastases of 4T1-T cells. Short hairpin (sh) RNA molecules are used that target Renilla luciferase (REN), SerpinE2 at two sites and SLPI at two sites.

FIG. 1E shows the selective ability of clones E and T to perform VM in vivo.

FIG. 1F shows the effect of SerpinE2 or SLPI knockdown on VM in vivo.

FIG. 1G are gel photographs showing the results of an in vitro Matrigel assay for VM. Only VM clones 4T1-E and 4T1-T can form tube structures when plated on Matrigel. All data from ref. 7.

FIG. 2 is a schematic representation of SerpinE2 and SLPI constructs for add-back experiments. In each case the HALO tag is separated from the gene by a Tobacco Etch Virus (TEV) protease cleavage site such that SerpinE2, SLPI or Aequorea coerulescens green fluorescent protein (acGFP) can be recovered from the HALO resin by TEV cleavage. acGFP serves as a control for expression of a HALO-tagged protein and serves as a negative control for interaction analysis by mass-spec. Any proteins found to interact with acGFP are removed from the analysis. LTR represents long terminal repeat sequences. Neo^{R} represents neomycin resistance gene. CMV represents a cytomegalovirus promoter. SP=Signal Peptide.

FIGS. 3A-3C shows that VM clones up-regulate secreted/extra-cellular factors that are necessary for maintenance of the VM state.

FIG. 3A shows enriched gene ontology terms of VM clones vs all clones, plotted is the negative log¹⁰ of the FDR corrected p value.

FIG. 3B shows representative 4× images of 4T1-T^{VM} cells treated as indicated.

FIG. 3C shows a quantification of 3 independent experiments, bars are mean +/− SEM.

FIGS. 4A-4I shows that IRE1 restrains secreted factors that are required for VM and metastasis.

FIG. 4A shows the RNA expression of ER stress/UPR regulators in basal-claudin-low breast cancer patients with or without relapse.

FIG. 4B shows a low level of IRE1 is associated with shorter relapse-free survival.

FIGS. 4C and 4D show suppression of IRE1 with RNAi enhances metastasis.

FIGS. 4E and 4F show that IRE1 inhibition augments, and activation represses, VM tubulogenesis.

FIG. 4G shows that IRE1 inhibition augments VM through secreted factors.

FIGS. 4H and 4I show that IRE1 regulates SLPI mRNA levels and stability.

FIGS. 5A-5F shows that tumor cell IRE1 regulates genes that promote vascular development.

FIG. 5A shows volcano plots of gene expression changes, as measured by polyA RNA-Seq, upon IRE1 inhibition or activation with Tunicamycin. Y axis is log² fold change mRNA levels and X axis is the negative log¹⁰ of the corrected p-value. Red points highlight significant changes with a p value below 0.05.

FIG. 5B shows a number of significantly changed genes and their overlap in different conditions.

FIG. 5C shows a Gene Ontology (GO) analysis of enriched terms in genes that are significantly down-regulated with IRE1 inhibition and significantly up-regulated with IRE1 activation.

FIG. 5D shows Gene Ontology (GO) analysis of enriched terms in genes that are significantly up-regulated with IRE1 inhibition and significantly down-regulated with IRE1 activation.

FIGS. 5E and 5F show Gene Set Enrichment Analysis (GSEA) of a rank ordered list of mean log² fold change values of IRE1 inhibition and the inverse log² fold change in Tunicamycin treatment.

FIGS. 6A-6E show the functional identification of VM-promoting IRE1-target mRNAs.

FIG. 6A shows a heatmap of loge fold changes in mRNA levels of genes that were significantly up-regulated by IRE1 inhibition and significantly down-regulated by IRE1 activation (with Tunicamycin) ranked by their loge fold change in VM clones (vs all other clones) and lung metastases (vs the primary tumor).

FIG. 6B shows the effect of MGP knockdown on the tube forming ability of 4T1-T^{VM} cells.

FIG. 6C shows the effect of ANK (ANKH human gene symbol) on the tube forming ability of 4T1-T^{VM} cells.

FIG. 6D shows the change in mRNA stability of MGP, ANK and SLPI upon IRE1 inhibition as measured by Actinomycin D run-off assays. Suggesting that these mRNAs are targets of RIDD (regulated IRE1-dependent mRNA decay).

FIG. 6E shows levels of MGP and ANKH mRNAs in basal/Claudin-low breast cancer patients that did or did not relapse with metastatic disease.

FIGS. 7A-7I shows that tumor cells co-opt a master endothelial transcription factor to drive VM gene expression and metastasis.

FIG. 7A shows the expression of all transcription factors from the GO database in VM clones relative to all other clones is shown.

FIG. 7B shows the expression of FOXC2 in cells derived from the primary breast tumor or lung metastases.

FIG. 7C shows the effect of FOXC2 over-expression on by human MDA-MB-231^{VM} cells.

FIG. 7D is a graph showing the effect of short hairpin (sh) RNA molecules that target Renilla luciferase as a negative control (REN) or one of two sites on FOXC2 (#1 and #2) on the tube forming ability of 4T1-T^{VM} cells.

FIG. 7E shows a Gene Set Enrichment Analysis (GSEA) of the top 100 FOXC2 target genes from GSE44335 and their enrichment in genes that go up in VM clones (left panel) or Lung metastases (right panel).

FIG. 7F show enriched GO terms in FOXC2 over-expressing HMLE cells relative to VM clones and Lung metastases.

FIG. 7G shows relapse-free survival of basal/claudin-low breast cancer patients by FOXC2 expression.

FIG. 7H shows GSEA signed P values (+ve enriched, −ye depleted) for FOXC2 and EMT TFs in VM clones vs all other clones.

FIG. 7I shows Tissue-Specific Expression Analysis (TSEA). Analysis of target genes of FOXC2 or EMT transcription factor target genes induced in normal epithelial cells for enrichment of tissue-specific genes. FOXC2 induces blood vessel-specific genes in epithelial cells suggesting an endothelial transition.

FIG. 8A is an overview of the RNAi screen for druggable regulators of FOXC2 function showing a schematic overview of FOXC2 reporter engineering. MEF2C, a key FOXC2 target in endothelium is tagged with mCherry at the endogenous locus using CRISPR/Cas9.

FIG. 8B is a schematic of the FACS-based RNAi screening strategy, the barcode vector that was used to generate 4T1-T^{VM} cells contains GFP and the reporter is mCherry. Cells are sorted into GFP/mCherry double positive population (background) and a GFP-high/mCherry-low population which represents “hits” whose knockdown suppresses reporter expression.

FIG. 9A-9D shows that VM-related genes are associated with poor response to Bevacizumab in patients.

FIG. 9A is a schematic overview of clinical trial outlined in Mehta et al., (2016) is shown.

FIG. 9B shows a GSEA demonstrating enrichment of Bev resistance genes in IRE1-regulated genes.

FIG. 9C shows a GSEA demonstrating enrichment of Bev resistance genes in FOXC2-regulated genes.

FIG. 9D shows additional data suggesting that drug regimens that impede VM in combination with angiogenesis inhibition improve tumor responses to anti-angiogenic agents showing the results of treating 4T1-TVM-derived tumors with Vehicle (Veh:Veh), Tunicamycin alone (Veh:Tunic), Sunitinib alone (Sunit:Veh, anti-angiogenic kinase inhibitor), or a combination of Tunicamycin and Sunitinib (Sunit: Tunic) using regimens as indicated. Tumor volumes were measured by bioluminescence prior to and after cessation of therapy. Shown are bioluminescent tumor volumes normalized to pre-treatment and vehicle treated tumors at day 6 after initiation of therapy. 4T1-TVM tumors are resistant to Sunitinib and are sensitized to Sunitinib by inhibiting VM in combination by using Tunicamycin as an IRE1 activator.

### DETAILED DESCRIPTION

Therapeutic compositions and methods are described for coordinating the inhibition of tumor vascularization and the inhibition or repression of vasculogenic mimicry, including for the treatment of cancers. The data provided in the examples below supports small molecule targeting of VM in combination with anti-angiogenic therapy. In another embodiment, a method is provided that uses a VM-based gene signature as a bio-marker for monitoring response to anti-angiogenic therapy, and/or to identify sub-sets of patients for whom combination anti-VM/anti-angiogenic therapy is beneficial.

### I. Definitions and Components of the Compositions and Methods

In the descriptions of the compositions and methods discussed herein, the various components are defined by use of technical and scientific terms having the same meaning as commonly understood by one of ordinary skill in the art to which this invention belongs and by reference to published texts. Such texts provide one skilled in the art with a general guide to many of the terms used in the present application. The definitions contained in this specification are provided for clarity in describing the components and compositions herein and are not intended to limit the claimed invention.

The terms “subject”, “patient”, or “mammalian subject”, as used herein include primarily humans, but can also be extended to include a veterinary or farm animal, a domestic animal or pet, and animals normally used for clinical research. In one embodiment, the subject of these methods and compositions is a human. Still other suitable mammalian subjects include, without limitation, murine, rat, canine, feline, porcine, bovine, ovine, and others.

The term “neoplastic disease”, “cancer” or “proliferative disease” as used herein refers to any disease, condition, trait, genotype, or phenotype characterized by unregulated or abnormal cell growth, proliferation, or replication. The abnormal proliferation of cells may result in a localized lump or tumor, be present in the lymphatic system, or may be systemic. In one embodiment, the neoplastic disease is benign. In another embodiment, the neoplastic disease is pre-malignant, i.e., potentially malignant neoplastic disease. In a further embodiment, the neoplastic disease is malignant, i.e., cancer. In still a further embodiment the neoplastic disease may be caused by viral infection. In one embodiment, the neoplastic disease is a cancer, such as an epithelial cancer.

In various embodiments of the methods and compositions described herein, the cancer can include, without limitation, breast cancer, lung cancer, prostate cancer, colorectal cancer, brain cancer, esophageal cancer, stomach cancer, bladder cancer, pancreatic cancer, cervical cancer, head and neck cancer, ovarian cancer, hepatocellular carcinoma (liver cancer), anal cancer, penile cancer, vulvar cancer, vaginal cancer, melanoma, leukemia, myeloma, lymphoma, glioma, and multidrug resistant cancer. In another embodiment, the neoplastic disease is Kaposi's sarcoma, Merkel cell carcinoma, multicentric Castleman's disease, primary effusion lymphoma, tropical spastic paraparesis, adult T-cell leukemia, Burkitt's lymphoma, Hodgkin's lymphoma, post-transplantation lymphoproliferative disease, nasopharyngeal carcinoma, pleural mesothelioma, osteosarcoma, ependymoma and choroid plexus tumors of the brain, and non-Hodgkin's lymphoma. In still other embodiments, the cancer may be a systemic cancer, such as leukemia. In one aspect, as exemplified, the cancer is a human glioblastoma. In another aspect, the cancer is a prostate adenocarcinoma. In still another embodiment, the cancer is a lung adenocarcinoma. In one embodiment, the cancer is non-small cell lung adenocarcinoma (NSCLC). In another embodiment, the cancer is squamous cell carcinoma. In another embodiment, the cancer is liver cancer. In another embodiment, the cancer is a breast cancer, such as, without limitation, breast adenocarcinoma. In yet a further embodiment, a cancer as referred to herein is a condition in which the subject's cancer or tumor is, or becomes over a period of time, refractory to treatment with anti-angiogenic therapy.

By the term “anti-angiogenic compound”, “anti-angiogenic therapy” or “anti-angiogenic therapeutic composition” as described herein is meant treatment with or use of any therapeutic agent that blocks or inhibits angiogenesis, inhibits blood vessel growth, or disrupts or removes angiogenic vessels either in vitro or in vivo. These compounds or compositions can cause tumor regression in various types of neoplasia (including benign neoplasia) or cancer. Known therapeutic candidates include naturally occurring angiogenic inhibitors, including without limitation, angiostatin, endostatin, and platelet factor-4. In another embodiment therapeutic candidates include, without limitation, specific inhibitors of endothelial cell growth, such as TNP-470, thalidomide, and interleukin-12. Still other anti-angiogenic agents include those that neutralize angiogenic molecules, such as including without limitation, antibodies to fibroblast growth factor or antibodies to vascular endothelial growth factor or antibodies to platelet derived growth factor or antibodies or other types of inhibitors of the receptors of EGF, VEGF or PDGF.

In other embodiments, antiangiogenic agents include without limitation suramin and its analogs, and tecogalan. In other embodiments, anti-angiogenic agents include without limitation agents that neutralize receptors for angiogenic factors or agents that interfere with vascular basement membrane and extracellular matrix, including, without limitation, metalloprotease inhibitors and angiostatic steroids. Another group of anti-angiogenic compounds includes, without limitation, anti-adhesion molecules, such as antibodies to integrin alpha v beta 3. Still other anti-angiogenic compounds or compositions, include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound is an antibody to VEGF, such as Avastin®/bevacizumab (Genentech).

FOXC2 or Forkhead box protein C2 (also known as MFH1) is a transcriptional activator that belongs to a large family of nuclear transcription factor proteins sharing a common forkhead/winged helix DNA binding domain. The human mRNA sequence for FOXC2 is found in the NCBI database as NM_005251.2; the protein sequence is published as NCBI database accession number NP-005242.1. This gene has been implicated in a wide number of cellular processes, e.g., as a regulator of epithelial to mesenchymal transition (EMT) and stem cell properties, including tumor-initiation capacity, metastatic competence, and chemotherapy resistance, tumor recurrence, triple-negative breast cancer (TNBC) progression and human lymphedemia-distichiasis syndrome, and tumor metastasis, and adipocyte morphogenesis. In addition, the transcriptional activity of FoxC2 influences expression of cytokine receptors such as CXCR4. See, e.g., Pietilä M, et al. FOXC2 regulates the G2/M transition of stem cell-rich breast cancer cells and sensitizes them to PLK1 inhibition. Scientific Reports. April 2016; 6:23070. doi:10.1038/srep23070, and references cited therein.

By the term “FOXC2 pathway targets” is meant to include, without limitation, any gene or encoded protein, which in cooperation with FOXC2, operates to cause, enhance, or increase VM when FOXC2 is activated or its expression increased. Such targets include, without limitation, one or more of the 25 interacting proteins identified in the STRING Interaction Network, version 10.5, namely, NDAC2, PPP2R2A, FMN1, PPP2R1A, RPS6KB1, PIN1, RPS6KA5, SGK3, RPS6KA6, AKT3, AKT1, STK32B, STK32C, RPS6KB2, PPP2R1A, PPP2R2A, RPS6KA4, FMN1, C5orf24, SGK2, SGK1, ZBTB34, RPS6KA3, AKT2, STK32A, RPS6KA2, HDAC2 and RPS6KA1. Such targets also include one or more of the transcriptional targets of the FOXC2 pathway that play key roles in vasculature development and metastasis, namely, MEF2C, SERPINE2, SLPI, GREM1, TMEM100, SERPINE1, CYP1B1, ANGPTL4, FGF2, PRKCA, PRKD1, ITGA5, GATA6, DDAH1, ADM, HMOX1, HIPK2, CCBE1, IL8, WNTSA, PTK2B, ECM1, HIF1A, SRPX2, TBXA2R, HSPB1, SPHK1, HGF, RAPGEF2, C3AR1, HDAC9, C5AR1, PDGFB, MTDH, RRAS, RHOB, SIRT1, CIB1, CCL5, ERAP1, C19ORF10, BTG1, PIK3R6, PLCG1, EGR1, ITGB2, GATA4, PHACTR1, RCAN2, SOBP, VCAN, FRY, FAM129A, GLIPR1, OSR1, NOV, EPS8, VIM, SDC2, COL6A2, WWTR1, TSC22D1, ENO2, ABI3BP, FOXL1, VASN, MYLK, PPP1R3C, DOCK10, KANK2, FN1, ANGPT1, LGALS3BP, CAMK1D, SOD3, CXXC5, CSGALNACT1, PNRC1, HTRA3, SDC3, SPP1, PLSCR4, ICAM1, TSPAN15, OSMR, KDELR3, TRIOBP, GBP4, ANGPTL2, TRIB2, and SLC15A3.

As used herein, the phrase “inhibits FOXC2 ” means that the expression or activity of the FOXC2 gene or level of RNA molecule encoding it is down-regulated, or less than that observed, in the absence of the selected FOXC2 modulator therapeutic reagent, with the result that vascular mimicry (VM) of a subject's tumor is inhibited, disrupted, or repressed. In one embodiment, this inhibition of VM co-operates with the anti-angiogenic effect of an anti-angiogenic compound, such as an anti-VEGF antibody. In another embodiment, this inhibition of VM synergizes with the anti-angiogenic effect of an anti-angiogenic compound, such as an anti-VEGF antibody. In one embodiment, this inhibition of VM co-operates with both the anti-angiogenic effect of an anti-angiogenic compound and the VM inhibiting effect of the IRE1 modulator. In another embodiment, this inhibition of VM synergizes with both the anti-angiogenic effect of the anti-angiogenic compound, and the VM inhibitory effect of the IRE1 modulator. In another embodiment, this inhibition of VM operates to prevent re-vascularization by VM of the tumor after the anti-angiogenic compound, such as an anti-VEGF antibody, reduces the normal vascularization of the tumor.

As used herein, the phrase “inhibits the FOXC2 pathway” or “inhibits a FOXC2 pathway target” means that the effect of a FOXC2 modulator on the expression or activity of the target RNA molecules encoding one or more target protein or protein subunits or peptides of the FOXC2 pathway up-regulates or down-regulates the target, such that the expression, level, or activity is greater than or less than that observed in the absence of the FOXC2 modulator therapeutic reagent, with the result that vascular mimicry (VM) of a subject's tumor is inhibited, disrupted or repressed. It should be understood that in one embodiment, certain FOXC2 pathway targets behave similarly to FOXC2, i.e., one target may be directly inhibited or its activity suppressed to achieve VM inhibition in a manner parallel to that of FOXC2. In another embodiment, depending upon the FOXC2 pathway target and its relationship to FOXC2 (i.e., FOXC2 expression may inhibit the target), the target may be directly activated or its activity enhanced (i.e., in a manner opposite to FOXC2) by the modulator to achieve VM inhibition. In one embodiment, this inhibition of VM co-operates with the anti-angiogenic effect of an anti-angiogenic compound, such as an anti-VEGF antibody. In another embodiment, this inhibition of VM synergizes with the anti-angiogenic effect of an anti-angiogenic compound, such as an anti-VEGF antibody. In another embodiment, this inhibition of VM operates to prevent re-vascularization by VM of the tumor after the anti-angiogenic compound, such as an anti-VEGF antibody, reduces the normal vascularization of the tumor.

As used herein, a “FOXC2 or FOXC2 pathway modulator” or “therapeutic compounds that inhibit FOXC2 or the FOXC2 pathway” refer to a therapeutic reagent, compound or composition that directly inhibits FOXC2 expression or activity so as to inhibit, disrupt or repress vascular mimicry, or directly inhibits a FOXC2 pathway target's expression or activity so as to inhibit, disrupt or repress vascular mimicry. These same phrases are also used to refer to a therapeutic reagent, compound or composition that directly activates or enhances a FOXC2 pathway target's expression or activity so as to inhibit, disrupt or repress vascular mimicry. In certain examples, therefore, FOXC2 modulators are therapeutic compounds that inhibit FOXC2 or the FOXC2 pathway, including without limitation, antibodies for FOX C2 or an associated pathway target, such as those provided by R&D Systems (MAB5044), Novus Biologicals Antibodies for FOXC2 (NB100-1269), ThermoFisher Scientific (MA5-17077), etc. Other inhibitors include shRNA, siRNA or RNAi sequences directed to FOXC2 or one of the “parallel-acting” targets (see, e.g., the FOXC2 directed inhibitors available from, e.g., Origene, Rockville, Md. or SantaCruz Biotechnology, Inc.; or ViGene Biosciences) or CRISPR/Cas guide systems that are commercially available or may be readily developed. Other FOXC2 pathway modulators directly activate certain FOXC2 pathway targets that are normally inhibited by FOXC2 expression or activity.

Additionally, small chemical compounds, such as, p38 MAPK inhibitors (including but not limited to, SB203580, AL 8697, AMG 548, BIRB 796, CMPD-1, DBM 1285 dihydrochloride, EO 1428, JX 401, ML 3403, Org 48762-0, PH 797804, RWJ 67657, SB 202190, SB 203580, SB 203580 hydrochloride, SB 239063, SB 706504, SCIO 469 hydrochloride, SKF 86002 dihydrochloride, SX 011, TA 01, TA 02, TAK 715, VX 702, VX 745 and p38 MAPK Inhibitor Tocriset™. See, e.g., www.tocris.com/pharmacology/p38-mapk), Cdk/Cdk5 inhibitors (including but not limited to, (R)-CR8, Aminopurvalanol A, Arcyriaflavin A, AZD 5438, BMS 265246, BS 181 dihydrochloride, CGP 60474, CGP 74514 dihydrochloride, CVT 313, (R)-DRF053 dihydrochloride, Flavopiridol hydrochloride, 10Z-Hymenialdisine, Indirubin-3′-oxime, Kenpaullone, NSC 625987, NSC 663284, NSC 693868, NU 2058, NU 140, Olomoucine, [Ala⁹²]-p16 (84-103), PD 0332991 isethionate, PHA 767491 hydrochloride, Purvalanol A, Purvalanol B, Ro 3306, Roscovitine, Ryuvidine, Senexin A, SNS 032 and SU 9516. See, e.g., www.tocris.com/pharmacology/cyclin-dependent-protein-kinases), PDGFR inhibitors (including but not limited to, Imatinib meseylate; Toceranib; Sunitinib malate; SU 6668; SU 16f; PD 166285 dihydrochloride; KG 5; GSK 1363089; DMPQ dihydrochloride; CP 673451; AP 24534; AG 18; and AC 710. See, e.g., www.tocris.com/pharmacology/pdgfr), PKA inhibitors (including but not limited to, H89 dichloride; Fasudil hydrochloride; cGMP Dependent Kinase Inhibitor Peptide; KT 5720; PKA inhibitor fragment (6-22) amide; PM (5-24); PM 14-22 amide, myristoylated; and cAMP antagonist, e.g., cAMPS-Rp, triethylammonium salt. See, e.g., www.tocris.com/pharmacology/protein-kinase-α), PKD inhibitors (including but not limited to, CID 755673, CID 2011756, CRT 0066101, and kb NB 142-70. See, e.g., www.tocris.com/pharmacology/protein-kinase-d), PI3K inhibitors (including but not limited to, PI 3-Kβ inhibitor, e.g., AZD 6482; PI 3-kinase inhibitors, e.g., A66, AS 252424, AS 605240, BAG 956, CZC 24832, ETP 45658, GSK 1059615; LTURM 36, LY 294002 hydrochloride, 3-Methyladenine, PI 103 hydrochloride, PI 3065, PI 828, PP 121, Quercetin, STK16-IN-1, TG 100713, TGX 221, Wortmannin; KU 0060648; LY 303511; PF 04691502; and PF 05212384. See, e.g., www.tocris.com/pharmacology/pi-3-kinase), MET inhibitors and MET kinase inhibitors (including but not limited to, Crizotinib, GSK 1363089, K 252a, Norleual, PF 04217903 mesylate, PHA 665752, SGX 523, SU 11274, SU 5416, and XL 184. See, e.g., www.tocris.com/pharmacology/met-receptors), CAMK inhibitors (including but not limited to, CaM kinase III inhibitors, e.g., A 484954, NH 125; CaM kinase II inhibitors, e.g., KN93 phosphate, KN 93, Arcyriaflavin A, Autocamtide-2-related inhibitory peptide, Autocamtide-2-related inhibitory peptide, myristoylated, KN-62; and CaM kinase inhibitor, e.g., STO-609 acetate. See, e.g., www.tocris.com/pharmacology/cam-kinase), FGFR inhibitors (including but not limited to, PD161570, AP 24534, FIIN 1 hydrochloride, PD 166285 dihydrochloride, PD 173074, SU 5402, and SU 6668. See, e.g., www.tocris.com/pharmacology/fgfr), and/or blocking antibodies against the above targets or their ligands may be useful as modulators of this pathway. IRE1, the transmembrane protein kinase inositol-requiring enzyme 1, is encoded by ERN1, the endoplasmic reticulum to nucleus signaling). The encoded protein contains two functional catalytic domains, a serine/threonine-protein kinase domain and an endoribonuclease domain. The human mRNA sequence for IRE1/ERN1 is found in the NCBI database as Gene ID 2081, NM_001433.4. The protein sequence is published as NCBI database accession number NP_001424.3. This protein functions as a sensor of unfolded proteins in the endoplasmic reticulum (ER) and triggers an intracellular signaling pathway termed the unfolded protein response (UPR). The UPR is an ER stress response that is conserved from yeast to mammals and activates genes involved in degrading misfolded proteins, regulating protein synthesis, and activating molecular chaperones. IRE1 suppresses mRNAs encoding secreted proteins to relieve overloading of the ER by secretory proteins in addition to mediating the splicing and activation of the stress response transcription factor X-box binding protein 1 (XBP1).

By the term “IRE1 pathway targets” is meant to include, without limitation, any gene or encoded protein, which in cooperation with IRE1, operates to decrease or inhibit VM when IRE1 is activated or its expression increased and/or when the activity of its target genes is inhibited or diminished. Such targets include, without limitation, one or more of the 25 interacting proteins identified in the STRING Interaction Network, version 10.5, namely, RB1CCA, XBP1, CCND1, PYCARD, CDK7, DERL1, MNAT1, CCND2, GTF2H1, GTF2H2, ERCC3, PYDC1, ACADB, ACACA, AKAP4, ERC1, BCCIP, CCND3, CCNY, PHKA2, ERCC2, DERL3, ATG13, GTF2H3, and PHKG2, among others. Such targets may also include without limitation, targets that are repressed upon IRE1 activation identified by RNA-Seq that play key roles in vasculture development and metastasis, namely, MGP, RBP1, SLPI, SERPINE2, AQP1, SFRP1, ICAM1, ANK, COL6A1, PROS1, PLSCR4, HTRA3, DECR1, NEURL3, ZHX1, PFN2, DMP1, IL1R1, NOD1, PADI2, RBP2, GCHFR, SAMSN1, C1QTNF1, ABCG1, TFDP2, PAPLN, TNFRSF9, OAF, PLAT, TSLP, MEGF6, H2AFV, ADD2, PADI3, DUSP27, GSTT1, S100A4, DNAJC12, HSPB1, SCNSA, NOV, CTSH, PRKG2, NGEF, FSD1L, UGDH, FBLIM1, LIX1L, AKR1C13, LPXN, DUSP6, RNF130, PTGR1, TMOD2, CST3, ANKRD6, RTKN2, IL12RB1, LDHB, BEND5, GM10471, SPN, RAET1E, RIN2, PDE6D, GNB4, MCTP1, PER3, LHPP, CALR3, CADM1, ITGB2, GHR, CRIP1, MSRB2, EGR2, PAQR7 DOK1, ACSBG1, LEPROT, FAM131B, GPRIN3, COL16A1, GRAP, FKBP1B, GSTMS, KANK2, PSG17, PIK3CD, INF2, MYLK, EML1, TDRD7, ALDH7A1, FAM219A, SH3BGRL, FAM221A, FAM102B, FN1, MAGED2, NUSAP1, M1AP, CISH, TBC1D2B, ATPIF1, MGST3, CNP, XKR5, NEIL3, RALGPS2, MTCH1, CAND2, MEST, TMEM243, XRCC3, NINJ2, ECM1, CPNE3, RAF1, SEPN1, CHST12, NADSYN1, CX3CL1, CD82, CDHR1, PEAR1L, POLD4, NR2F1, FHL2, ATHL1, CDKN2AIPNL, RAET1D, SCARA3, PLSCR2, and CRTAP.

As used herein, the phrase “activates IRE1” means that the expression or activity of the IRE1 gene or level of RNA molecule encoding it is up-regulated or greater than that observed in the absence of a IRE1 modulator therapeutic reagent, with the result that vascular mimicry (VM) of a subject's tumor is inhibited, disrupted, or repressed. In one embodiment, this inhibition of VM co-operates with the anti-angiogenic effect of an anti-angiogenic compound, such as an anti-VEGF antibody. In another embodiment, this inhibition of VM synergizes with the anti-angiogenic effect of an anti-angiogenic compound, such as an anti-VEGF antibody. In one embodiment, this inhibition of VM co-operates with both the anti-angiogenic effect of an anti-angiogenic compound and the VM inhibiting effect of the FOXC2 modulator. In another embodiment, this inhibition of VM synergizes with both the anti-angiogenic effect of the anti-angiogenic compound, and the VM inhibitory effect of the FOXC2 modulator. In another embodiment, this inhibition of VM operates to prevent re-vascularization by VM of the tumor after the anti-angiogenic compound, such as an anti-VEGF antibody, reduces the normal vascularization of the tumor.

As used herein, the phrase “activates IRE1” or “inhibits an IRE1 pathway target” means that the effect of an IRE1 modulator on the expression or activity of the target RNA molecules encoding one or more target protein or protein subunits or peptides of the IRE1 pathway is up regulated or down regulated such that the expression, level, or activity is greater than or less than that observed in the absence of the IRE1 modulator therapeutic reagent, with the result that vascular mimicry (VM) of a subject's tumor is inhibited, disrupted or repressed. It should be understood that in one embodiment, certain IRE1 pathway targets behave similarly to IRE1, i.e., the target, like IRE1 itself, may be directly activated or its activity enhanced to achieve VM inhibition in a manner parallel to that of IRE1. In another embodiment, depending upon the IRE1 pathway target and its relationship to IRE1 (i.e., IRE1 expression may inhibit the target), the target may be directly inhibited or its activity suppressed (i.e., in a manner opposite to IRE1) by the IRE1 modulator to achieve VM inhibition. In one embodiment, this inhibition of VM co-operates with the anti-angiogenic effect of an anti-angiogenic compound, such as an anti-VEGF antibody. In another embodiment, this inhibition of VM synergizes with the anti-angiogenic effect of an anti-angiogenic compound, such as an anti-VEGF antibody. In one embodiment, this inhibition of VM co-operates with both the anti-angiogenic effect of an anti-angiogenic compound and the VM inhibiting effect of the FOXC2 modulator. In another embodiment, this inhibition of VM synergizes with both the anti-angiogenic effect of the anti-angiogenic compound, and the VM inhibitory effect of the FOXC2 modulator. In another embodiment, this inhibition of VM operates to prevent re-vascularization by VM of the tumor after the anti-angiogenic compound, such as an anti-VEGF antibody, reduces the normal vascularization of the tumor.

As used herein, an “IRE1 or IRE1 pathway modulator” or “therapeutic compounds that activate IRE1 or the IRE1 pathway” refer to a therapeutic reagent, compound or composition that directly activates IRE1 expression or activity so as to inhibit, disrupt or repress vascular mimicry, or directly activates an IRE1 pathway target's expression or activity so as to inhibit, disrupt or repress vascular mimicry. Alternatively, an IRE1 pathway modulator refers to a therapeutic reagent, compound or composition that directly inhibits or reduces an IRE1 pathway target's expression or activity so as to inhibit, disrupt or repress vascular mimicry. In certain examples, therefore, therapeutic compounds that activate IRE1 include tunicamycin. Additionally, small molecule chemical compounds, such as thapsigagin, DTT, brefaldin A, bortezimib, acetaminophen, amiodarone, arsenic trioxide, Bleomycin, cisplatin, clozapine, olanzapine, cyclosporin, diclofenac, indomethacin, efavirenz, Proteasome inhibitors, zidovudine, sertraline, troglitazone, erlotinib, doxorubicin, and anitbodies directed against targets of the IRE1 pathway listed above, may also be useful as IRE1 modulators of this pathway.

Additionally, therapeutic compounds that inhibit an IRE1 pathway target that is normally inhibited when IRE1 itself is activated can include antibodies for that IRE1 pathway target, such as those provided by the same commercial entities referenced above for FOXC2 antibodies. Other “IRE1 modulators” therefore include shRNA, siRNA or RNAi sequences directed to one of those IRE1 targets that are activated when IRE1 is inhibited or CRISPR/Cas guide systems that are commercially available or may be readily developed.

As used herein for the described methods and compositions, the term “antibody” refers to an intact immunoglobulin having two light and two heavy chains or fragments thereof capable of binding to a FOXC2 protein or suitable FOXC2 pathway target or an IRE1 pathway target (that is inhibited when IRE1 is activated). Thus, by reference to an antibody includes a monoclonal antibody, a synthetic antibody, a recombinant antibody, a chimeric antibody, a humanized antibody, a human antibody, or a bi-specific antibody or multi-specific construct. The term “antibody fragment” as used herein for the described methods and compositions refers to less than an intact antibody structure having antigen-binding ability. Such fragments, include, without limitation, an isolated single antibody chain or an scFv fragment, which is a recombinant molecule in which the variable regions of light and heavy immunoglobulin chains encoding antigen-binding domains are engineered into a single polypeptide. Other scFV constructs include diabodies, i.e., paired scFvs or non-covalent dimers of scFvs that bind to one another through complementary regions to form bivalent molecules. Still other scFV constructs include complementary scFvs produced as a single chain (tandem scFvs) or bispecific tandem scFvs. Other antibody fragments include an Fv construct, a Fab construct, an Fc construct, a light chain or heavy chain variable or complementarity determining region (CDR) sequence, etc. Still other antibody fragments include monovalent or bivalent minibodies (miniaturized monoclonal antibodies) which are monoclonal antibodies from which the domains non-essential to function have been removed. In one embodiment, a minibody is composed a single-chain molecule containing one V_{L}, one V_{H} antigen-binding domain, and one or two constant “effector” domains. Linker domains connect these elements. In still another embodiment, the antibody fragments useful in the methods and compositions herein are “unibodies”, which are IgG4 molecules from with the hinge region has been removed.

By “pharmaceutically acceptable carrier or excipient” is meant a solid and/or liquid carrier, in in dry or liquid form and pharmaceutically acceptable. The compositions are typically sterile solutions or suspensions. Examples of excipients which may be combined with the anti-angiogenic compound, the FOXC2 modulator or IRE1 activator include, without limitation, solid carriers, liquid carriers, adjuvants, amino acids (glycine, glutamine, asparagine, arginine, lysine), antioxidants (ascorbic acid, sodium sulfite or sodium hydrogen-sulfite), binders (gum tragacanthin, acacia, starch, gelatin, polyglycolic acid, polylactic acid, poly-d,l-lactide/glycolide, polyoxaethylene, polyoxapropylene, polyacrylamides, polymaleic acid, polymaleic esters, polymaleic amides, polyacrylic acid, polyacrylic esters, polyvinylalcohols, polyvinylesters, polyvinylethers, polyvinylimidazole, polyvinylpyrrolidon, or chitosan), buffers (borate, bicarbonate, Tris-HCl, citrates, phosphates or other organic acids), bulking agents (mannitol or glycine), carbohydrates (such as glucose, mannose, or dextrins), clarifiers, coatings (gelatin, wax, shellac, sugar or other biological degradable polymers), coloring agents, complexing agents (caffeine, polyvinylpyrrolidone, β-cyclodextrin or hydroxypropyl-β-cyclodextrin), compression aids, diluents, disintegrants, dyes, emulsifiers, emollients, encapsulating materials, fillers, flavoring agents (peppermint or oil of wintergreen or fruit flavor), glidants, granulating agents, lubricants, metal chelators (ethylenediamine tetraacetic acid (EDTA)), osmo-regulators, pH adjustors, preservatives (benzalkonium chloride, benzoic acid, salicylic acid, thimerosal, phenethyl alcohol, methylparaben, propylparaben, chlorhexidine, sorbic acid, hydrogen peroxide, chlorobutanol, phenol or thimerosal), solubilizers, sorbents, stabilizers, sterilizer, suspending agent, sweeteners (mannitol, sorbitol, sucrose, glucose, mannose, dextrins, lactose or aspartame), surfactants, syrup, thickening agents, tonicity enhancing agents (sodium or potassium chloride) or viscosity regulators. See, the excipients in “Handbook of Pharmaceutical Excipients”, 5^{th} Edition, Eds.: Rowe, Sheskey, and Owen, APhA Publications (Washington, D.C.), 2005 and U.S. Pat. No. 7,078,053, which are incorporated herein by reference. The selection of the particular excipient is dependent on the nature of the compound selected and the particular form of administration desired.

Solid carriers include, without limitation, starch, lactose, dicalcium phosphate, microcrystalline cellulose, sucrose and kaolin, calcium carbonate, sodium carbonate, bicarbonate, lactose, calcium phosphate, gelatin, magnesium stearate, stearic acid, or talc. Fluid carriers without limitation, water, e.g., sterile water, Ringer's solution, isotonic sodium chloride solution, neutral buffered saline, saline mixed with serum albumin, organic solvents (such as ethanol, glycerol, propylene glycol, liquid polyethylene glycol, dimethylsulfoxide (DMSO)), oils (vegetable oils such as fractionated coconut oil, arachis oil, corn oil, peanut oil, and sesame oil; oily esters such as ethyl oleate and isopropyl myristate; and any bland fixed oil including synthetic mono- or diglycerides), fats, fatty acids (include, without limitation, oleic acid find use in the preparation of injectables), cellulose derivatives such as sodium carboxymethyl cellulose, and/or surfactants.

By “chemotherapeutic agent or therapy” is meant a drug or therapy designed for using in treating cancers. Examples of chemotherapeutics which may be utilized as described herein include, without limitation, cisplatin, carboplatin, 5-fluorouracil, cyclophosphamide, oncovin, vincristine, prednisone, rituximab, mechlorethamine, cyclophosphamide, ifosfamide, melphalan, chlorambucil, carmustine, lomustine, semustine, thriethylenemelamine, triethylene thiophosphoramide, hexamethylmelamine altretamine, busulfan, triazines dacarbazine, methotrexate, trimetrexate, fluorodeoxyuridine, gemcitabine, cytosine arabinoside, 5-azacytidine, 2,2′-difluorodeoxycytidine, 6-mercaptopurine, 6-thioguanine, azathioprine, 2′-deoxycoformycin, erythrohydroxynonyladenine, fludarabine phosphate, 2-chlorodeoxyadenosine, camptothecin, topotecan, irinotecan, paclitaxel, vinblastine, vincristine, vinorelbine, docetaxel, estramustine, estramustine phosphate, etoposide, teniposide, mitoxantrone, mitotane, or aminoglutethimide. Other anti-cancer therapies for use with the methods and compositions as described herein include non-chemical therapies. In one embodiment, the additional or adjunctive therapy includes, without limitation, radiation, acupuncture, surgery, chiropractic care, passive or active immunotherapy, X-ray therapy, ultrasound, diagnostic measurements, e.g., blood testing. In one embodiment, these therapies are utilized to treat the patient. In another embodiment, these therapies are utilized to determine or monitor the progress of the disease, the course or status of the disease, relapse or any need for booster administrations of the compounds discussed herein.

By “administering” or “route of administration” is delivery of the anti-angiogenic compound, FOXC2 modulator or IRE1 modulator, with or without a pharmaceutical carrier or excipient, or with or without another chemotherapeutic agent into the subject with cancer, the environment of the cancer cell or the tumor microenvironment of the subject. Conventional and pharmaceutically acceptable routes of administration include, but are not limited to, systemic routes, such as intraperitoneal, intravenous, intranasal, intravenous, intramuscular, intratracheal, subcutaneous, and other parenteral routes of administration or intratumoral or intranodal administration. In one embodiment, the route of administration is oral. In another embodiment, the route of administration is intraperitoneal. In another embodiment, the route of administration is intravascular. Routes of administration may be combined, if desired. In some embodiments, the administration is repeated periodically.

By “effective amount” is meant the amount or concentration (by single dose or in a dosage regimen delivered per day) of the anti-angiogenic compound, FOXC2 modulator and/or IRE1 modulator sufficient to retard, suppress or prevent the occurrence of vascularization to the tumor or cancer cell and simultaneously suppress vascular mimicry, while providing the least negative side effects to the treated subject. The amount of anti-angiogenic compound, FOXC2 modulator and/or IRE1 modulator for administration alone or in combination with an additional reagent, e.g., chemotherapeutic, antibiotic or the like can be determined with regard to the age, physical condition, weight and other considerations. In one embodiment, the effective amount(s)is an amount larger than that required when a anti-angiogenic compound is administered to inhibit angiogenesis of a tumor in a subject. In another embodiment, the effective amount of the anti-angiogenic compound is the same as that reported for its use as a sole therapeutic. In still another embodiment, the effective amount is that required to reduce or suppress vascularization of the tumor when administered in combination with the FOXC2 modulator or IRE1 modulator. In a further embodiment, the combination of the FOXC2 modulator and/or IRE1 modulator with the anti-angiogenic compound permits lower than usual amounts of any one of the three therapeutic reagents alone to achieve the desired therapeutic effect. In another embodiment, the combination of the anti-angiogenic compound with the FOXC2 modulator and/or IRE1 modulator and further with another chemotherapy treatment protocol permits adjustment of the additional protocol regimen to achieve the desired therapeutic effect. In one embodiment, the effective amount of the anti-angiogenic compound with the FOXC2 modulator and/or IRE1 modulator is within the range of 1 mg/kg body weight to 100 mg/kg body weight of each therapeutic agent in humans including all integers or fractional amounts within the range. In certain embodiments, the effective amount is at least 2, 5, 10, 15, 20, 25, 30, 35, 40, 45, 50, 55, 60, 65, 70, 75, 80, 85, 90, 95 or 100 mg/kg body weight, including all integers or fractional amounts within the range. In one embodiment, the above amounts represent a single dose of each therapeutic agent. In another embodiment, the above amounts define an amount(s) of each therapeutic agent to be delivered to the subject per day. In another embodiment, the above amounts define an amount delivered to the subject per day in multiple doses. In still other embodiments, these amounts represent the amount delivered to the subject over more than a single day.

“Control” or “Control subject” as used herein with reference to diagnostic methods refers to the source of the reference FOXC2 or IRE1 gene expression signatures or profiles to which the gene signature of the subject is being compared, as well as the particular panel of control subjects described herein. In one embodiment, the control or reference level is from a single subject. In another embodiment, the control or reference level is from a population of individuals sharing a specific characteristic, e.g., increasing VM or decreasing VM or no VM. In yet another embodiment, the control or reference level is an assigned value which correlates with the level of a specific control individual or population, although not necessarily measured at the time of assaying the test subject's sample. In one embodiment, the control subject or reference is from a patient (or population) having a non-cancerous nodule. In another embodiment, the control subject or reference is from a patient (or population) having a cancerous tumor.

“Sample” as used herein means any biological fluid or tissue that contains immune cells and/or cancer cells. The most suitable sample for use in this invention includes whole blood. Other useful biological samples include, without limitation, peripheral blood mononuclear cells, plasma, saliva, urine, synovial fluid, bone marrow, cerebrospinal fluid, vaginal mucus, cervical mucus, nasal secretions, sputum, semen, amniotic fluid, bronchoscopy sample, bronchoalveolar lavage fluid, and other cellular exudates from a patient having cancer. Still other samples include tissue from a tumor biopsy. Such samples may further be diluted with saline, buffer or a physiologically acceptable diluent. Alternatively, such samples are concentrated by conventional means.

By “change in expression” is meant an upregulation of one or more selected genes in comparison to the reference or control; a downregulation of one or more selected genes in comparison to the reference or control; or a combination of certain upregulated genes and down regulated genes.

In the context of the diagnostic compositions and methods described herein, reference to multiple gene targets in a gene signature or profile means any one or any and all combinations of the FOX2C or IRI-1 gene targets listed above, and including other genes that change expression during VM. For example, suitable gene expression profiles include profiles containing any number between at least 1 through at least about 500 genes that change expression during VM. In certain embodiment, A VM gene signature or gene profile is formed by at least 2, 5, 10, 15, 20, 25, 30, 35, 40, 45, 50, 55, 60, 65, 70, 75, 80, 85, 90, 95, 100, 125, 150, 200, 250, 300, 350, 400, 450 or 500 of the gene targets that change in expression during VM. See e.g., the targets identified herein and in the Figures.

The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The terms “a” or “an” refers to one or more. For example, “an expression cassette” is understood to represent one or more such cassettes. As such, the terms “a” or “an”), “one or more,” and “at least one” are used interchangeably herein.

As used herein, the term “about” means a variability of plus or minus 10% from the reference given, unless otherwise specified.

The words “comprise”, “comprises”, and “comprising” are to be interpreted inclusively rather than exclusively, i.e., to include other unspecified components or

The words “consist”, “consisting”, and its variants, are to be interpreted exclusively, rather than inclusively, i.e., to exclude components or steps not specifically recited.

As used herein, the phrase “consisting essentially of” limits the scope of a described composition or method to the specified materials or steps and those that do not materially affect the basic and novel characteristics of the described or claimed method or composition. Where ever in this specification, a method or composition is described as “comprising” certain steps or features, it is also meant to encompass the same method or composition consisting essentially of those steps or features and consisting of those steps or features.

### II. Compositions

In one embodiment, a therapeutic composition for the treatment or inhibition of tumor vacularization comprises the combination of an anti-angiogenic therapeutic compound and a therapeutic compound that inhibits or prevents vasculogenic mimicry or vascular mimicry (VM). In one embodiment, the therapeutic composition contains, in a suitable pharmaceutical carrier, an anti-angiogenic therapeutic compound, and a FOXC2 modulator therapeutic compound that inhibits the activity or pathway of the transcription factor FOXC2, thereby inhibiting or suppressing VM. As used throughout the specification and for simplicity, the exemplary anti-angiogenic therapeutic compound is referred to as an anti-VEGF antibody. In other embodiments, the term anti-VEGF antibody can be replaced with another anti-angiogenic compound identified above.

In another embodiment, the therapeutic composition contains in a suitable pharmaceutical carrier, an anti-angiogenic therapeutic compound, i.e., anti-VEGF antibody and a therapeutic compound that activates or enhances the activity or pathway of IRE1, i.e., an IRE1 activator.

In still another embodiment, the therapeutic composition contains in a suitable pharmaceutical carrier, an anti-angiogenic therapeutic compound, a therapeutic compound (i.e., a FOXC2 modulator) that inhibits the activity or pathway of the transcription factor FOXC2 and a therapeutic compound that activates or enhances the activity or pathway of IRE1 and inhibits the activity of the target genes of IRE1.

These compositions contain the two or three therapeutic components in effective amounts. The anti-VEGF antibody is present in an amount effective to suppress normal vascularization of a tumor present in a subject to be treated with the composition. If, present, the FOXC2 modulator is in an amount effective to inhibit the normal functioning of the FOXC2 pathway and suppress or prevent the occurrence of VM. If present, the IRE1 modulator is present in an amount effective to activate or overexpress the normal functioning of the IRE1 pathway and suppress or prevent the occurrence of VM. In compositions containing both the FOXC2 modulator and the IRI1 activator with the anti-VEGF antibody, the FOXC2 modulator and the IRI1 activator may be employed in effective amounts lower than those used when the inhibitor or activator is used alone (with the anti-VEGF).

The various components of the compositions are prepared for administration by being suspended or dissolved in a pharmaceutically or physiologically acceptable carrier In one embodiment, these at least two or all three components may be present in a pharmaceutical carrier in a single solution for simultaneous administration to the subject having cancer.

It is also anticipated that where desired, a therapeutic kit is provided that contains individually packaged effective amounts of the two (anti-VEGF antibody and at least one of the FOXC2 modulator or IRE1 modulator) or three (anti-VEGF antibody, FOXC2 modulator and IRE1 modulator) components. Such a kit is convenient for administration of each component separately and sequentially and can contain additional “booster” doses of any of the three components, where needed. Conventional kit components, such as packaging, additional pharmaceutical carriers, drug delivery devices and any adjunctive treatment modalities, may be included in the kit.

In yet another aspect, a composition or kit for diagnosing or evaluating the efficacy of cancer treatment in a mammalian subject includes multiple polynucleotides or oligonucleotides, wherein each polynucleotide or oligonucleotide hybridizes to a different gene, gene fragment, gene transcript or expression product in a patient sample, where each gene, gene fragment, gene transcript or expression product is selected from genes that are upregulated or down-regulated in the course of VM or in the course of treatment for VM. Such genes include the gene targets identified herein as FOXC2 or IRE-1 pathway targets, identified above. By evaluating the gene targets that change in expression during vascular mimicry in a cancer patient, a physician may assess the severity of the cancer and/or the success of the treatment described herein. In one embodiment of such a diagnostic composition, at least one polynucleotide or oligonucleotide is attached to a detectable label.

### III. The Methods

Any of the above-described compositions and/or kits with individual components may be employed in methods for the treatment or inhibition of tumor vacularization and/or the treatment of cancers. In one embodiment, a method for increasing the sensitivity of a tumor to anti-angiogenic therapy comprises treating a patient having a tumor with an anti-angiogenic therapeutic composition or compound and substantially simultaneously inhibiting vascular, or vasculogenic, mimicry (VM). Inhibition of VM comprises further administering at least one of a therapeutic compound that inhibits the activity or pathway of the transcription factor FOXC2; and a therapeutic compound that activates or enhances the activity or pathway of IRE1 and/or inhibits the activity of its target genes.

Thus in one embodiment, the method involves administering to a subject with a cancer the anti-VEGF antibody in a suitable pharmaceutical carrier. This method also involves administering a therapeutic compound that inhibits the activity or pathway of the transcription factor FOXC2 in an amount effective to suppress VM in a pharmaceutical carrier. Such administration can occur by a suitable route of administration and dosage depending upon the physical condition of the subject, and whether these components are being administered simultaneously in a single composition or sequentially.

Thus in another embodiment, the method involves administering to a subject with a cancer the anti-VEGF antibody in a suitable pharmaceutical carrier. This method also involves administering a therapeutic compound that activates or enhances the activity or pathway of IRE1 in an amount effective to suppress VM in a pharmaceutical carrier. Such administration can occur by a suitable route of administration and dosage depending upon the physical condition of the subject, and whether these components are being administered simultaneously in a single composition or sequentially.

Thus yet another embodiment, the method involves administering to a subject with a cancer the anti-VEGF antibody in a suitable pharmaceutical carrier. This method also involves administering a therapeutic compound that inhibits the activity or pathway of the transcription factor FOXC2 in an amount effective to suppress VM in a pharmaceutical carrier. This method also involves administering a therapeutic compound that activates or enhances the activity or pathway of IRE1 in an amount effective to suppress VM in a pharmaceutical carrier. Such administration can occur by a suitable route of administration and dosage depending upon the physical condition of the subject, and whether these components are being administered simultaneously in a single composition or sequentially.

Methods for determining the timing of frequency (boosters) of administration on one, two or all three of the components will include an assessment of disease response, including assessments of tumor size. In another embodiment, any of these above-receited methods further comprises administering to the subject along with the therapeutic agents, an adjunctive therapy, such as chemotherapy or radiation, or others as described above directed toward the cancer or tumor being treated.

Additional modifications of these methods includes changing the FOXC2 pathway target being treated with the FOXC2 pathway modulator (inhibitor or activator, as necessary) as defined above with each “booster” treatment or changing the IRE1 pathway target being treated with the IRE1 modulator as defined above with each “booster” treatment. In another embodiment, administration of the FOXC2 modulator and IRE1 modulator are alternated in the regimen. In still another embodiment, treatment steps can involve alternating or repeating the administration of FOXC2 modulators, wherein each treatment step is designed to directly effect a different or alternative FOXC2 pathway target or multiple FOXC2 pathway targets, simultaneously or sequentially. In still another embodiment, treatment steps can involve alternating or repeating the administration of IRE1 modulators, wherein each treatment step is designed to directly effect a different or alternative IRE1 pathway target, or multiple IRE1 pathway targets, simultaneously or sequentially. One of skill in the art can assemble any number of treatment regimens by alternating the two or three active components of the methods.

In still another embodiment, a method for the treatment or inhibition of tumor vacularization and/or for the treatment of a cancer comprises treating a patient having a tumor with an antibody to VEGF and substantially simultaneously inhibiting vascular, or vasculogenic, mimicry (VM). As described above, inhibition of VM comprises administering at least one of a therapeutic compound that inhibits the activity or pathway of the transcription factor FOXC2; and a therapeutic compound that activates or enhances the activity or pathway of IRE1 and/or inhibits the activity of its target genes. In one embodiment, the cancer is a breast cancer. In other embodiments, the cancer is any of those identified above.

In still another aspect, a method for diagnosing or evaluating cancer characterized by VM in a mammalian subject involves identifying changes in the expression of three or more genes in the sample of a subject, said genes selected from the gene targets identified herein; and comparing that subject's gene expression levels with the levels of the same genes in a reference or control. Changes in expression of such gene targets correlates with a diagnosis or evaluation of the progression of a cancer, e.g., breast cancer, characterized by characteristic gene target expression changes that occur with increasing VM. Alternatively, changes in expression of such gene targets correlates with a diagnosis or evaluation of the treatment of a cancer with angiogenic therapy coupled with anti-VM therapy as described herein, wherein successful treatment is characterized by gene target expression changes that occur with decreasing VM. The compositions and methods described herein provide the ability to distinguish the progress of vascular mimicry in a patient, by determining a characteristic RNA expression profile of the genes of the blood of a mammalian, preferably human, subject. The profile of certain genes upregulated or down-regulated during VM is compared with the profile of one or more subjects of the same class (e.g., patients having lung cancer or a non-cancerous nodule) or a control to provide a useful diagnosis.

Such methods of gene expression profiling include methods based on hybridization analysis of polynucleotides, methods based on sequencing of polynucleotides, and proteomics-based methods. The most commonly used methods known in the art for the quantification of mRNA expression in a sample include northern blotting and in situ hybridization; RNAse protection assays; nCounter® Analysis; and PCR-based methods, such as RT-PCR. Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

It should be understood that other modifications of these methods by selection of the components identified above may be readily performed using knowledge in the art coupled with the teachings in this specification.

The following examples, protocols and methods described in the examples are not considered to be limitations on the scope of the claimed invention. Rather this specification should be construed to encompass any and all variations that become evident as a result of the teaching provided herein. One of skill in the art understand that changes or variations can are made in the disclosed embodiments of the examples, and expected similar results can are obtained. For example, the substitutions of reagents that are chemically or physiologically related for the reagents described herein are anticipated to produce the same or similar results. All such similar substitutes and modifications are apparent to those skilled in the art and fall within the scope of the invention.

Additionally, we deploy a battery of state of the art technologies such as single cell RNA-Seq, CLIP, and CHIP-Seq, and we bring these technologies together in an innovative fashion to achieve a broad, yet specific, systems level understanding of VM. For example, we couple CRIPSR/Cas9-mediated genome editing to achieve epitope tagging of proteins at their endogenous loci, coupled with CHIP-Seq (for FOXC2), CLIP (for IRE1) or RNAi screening (for the FOXC2-reporter at the MEF2C locus). These approaches not only generate the necessary tools for our research but also provide tools and resources for the broader scientific community. For example, IRE1 CLIP coupled with RNA-Seq in IRE1 loss- and gain-of-function models provide the first transcriptome-wide map of IRE1 protein-RNA interactions, facilitate motif discovery, and identify cleavage sites and their functional consequences.

### EXAMPLE 1

### Methods and Materials—In Vivo Procedures

We endeavor to use non-animal model systems wherever possible, such as cell lines, in vitro and computational approaches, we are studying processes which are fundamentally related to interactions between the host and the tumor such as metastasis and response to therapy which require a whole organism to yield meaningful insights. Moreover, metastasis is a complex process which involves multiple hurdles that tumor cells must overcome to form occult secondary metastases such as invasion through the primary tumor, entry, and survival into the blood stream, exit from the blood stream and colonization at distant sites. Each of these steps is influenced by anatomical, immune, and environmental factors that only exist in an intact organism.

Mice are chosen for our experiments as they are the organism from which 4T1 cells were derived, facilitating transplantation into immune-competent hosts. Moreover, some drugs that we use in our studies specifically recognize murine proteins as such experiments using such drugs necessitate the use of mice.

Balb/C and Balb/C nudes are used to transplant tumor cells of human or mouse origin into the mammary fat pad. Following tumor formation animals are used as efficiently as possible to reduce the total numbers of animals used. We measure primary tumor volumes, circulating tumor cells (via PCR) and secondary metastases (Lung, Brain, Bone metastases).

All procedures are performed in the most humane fashion to minimize pain and stress for the animals. The procedures performed do not involve survival surgery. When required for tumor cell injections etc. Isoflurane anesthesia are used, the most commonly used inhalational anesthetic in animal care facilities. All animal experiments are performed at the Rockefeller University Comparative Bioscience Center (CBC) which provides a comprehensive program of animal care in support of the in vivo research.

Animals are screened on a regular basis for any signs of pain or distress. Animals exhibiting such signs are euthanized. Euthanasia is performed with CO₂. This method is consistent with the recommendations of the American Veterinary Medical Association (AVMA) Guidelines for the Euthanasia of Animals.

### EXAMPLE 2

### The Serine Protease Inhibitors Serpine2 and SLPI Regulate VM

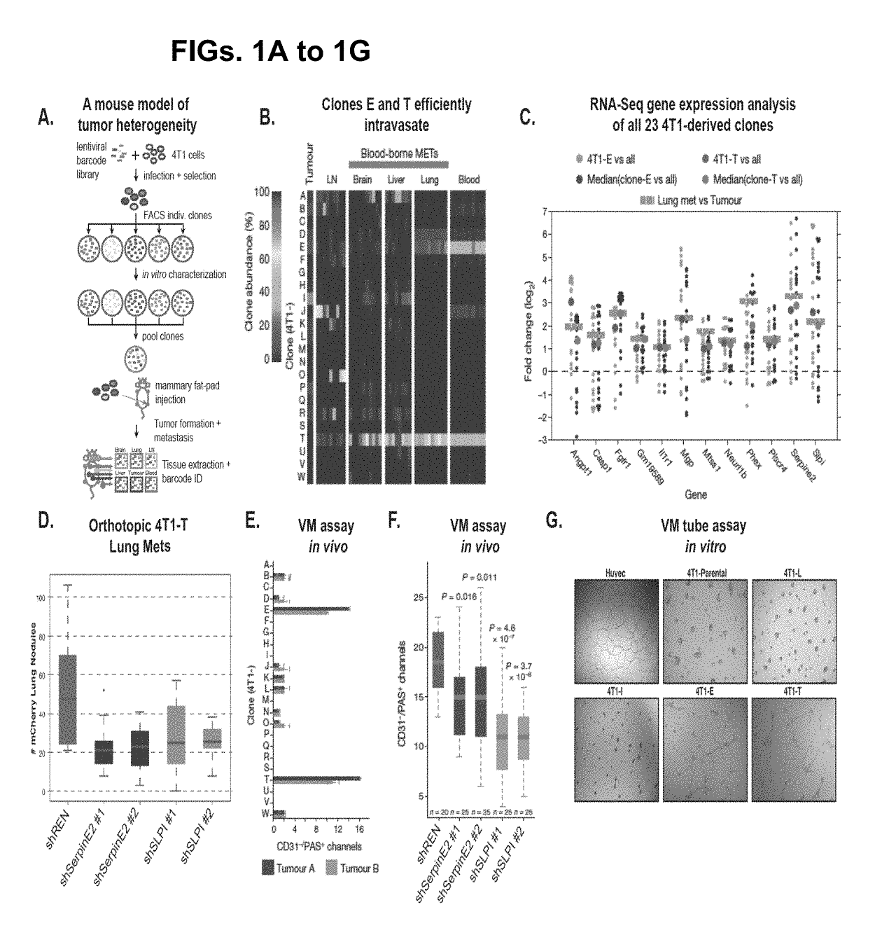

Recently, using genetic tracking of clonal lineages derived from the same parental population (FIGS. 1A and 1B), we have identified sub-clones of breast cancer cells that are competent to perform VM (FIGS. 1E-1G)⁷. By comparing gene expression between VM-competent and incompetent sub-clones we identified several putative mediators of VM (FIG. 1C), two of which we functionally validated in vitro and in vivo (FIGS. 1F-G). Through manipulation of these novel VM mediators, SerpinE2 and SLPI, we discovered an essential role for VM in metastasis (FIG.1D) by facilitating tumor cell intravasation (entry into the bloodstream)⁷. These data not only established the importance of VM for metastasis but also identified and validated the first bona fide drivers of the VM phenotype. However, the targets through which SerpinE2 and SLPI act, the precise mechanisms through which otherwise epithelial cells differentiate into endothelial-like cells, and whether VM causally underlies resistance to anti-angiogenic therapy are unknown. Here, we delve into the molecular details of discrete steps of VM by ascertaining the role of transcriptional control in the initiation of VM, the role of secretory programs, including SerpinE2 and SLPI, in the maintenance of VM, and the downstream consequences of these programs in disease.

Using a combination of cell biology, genetics, biochemistry, and mass spectrometry approaches, we determine the underlying mechanisms by which SerpinE2 and SLPI promote VM, including identifying their direct protein targets and mechanisms of action and testing whether they work in a solely autocrine or also paracrine fashion. Broadly we examine the consequences of SerpinE2 and SLPI up-regulation on VM including the locale where they exert their effects (i.e. intra or extra-cellular), their targets, and the downstream effects, utilizing a combination of cell biology, biochemistry, and mass-spectrometry.

A. Determine Whether Secretion is Necessary for SerpinE2 and SLPI to Influence VM and Metastasis.

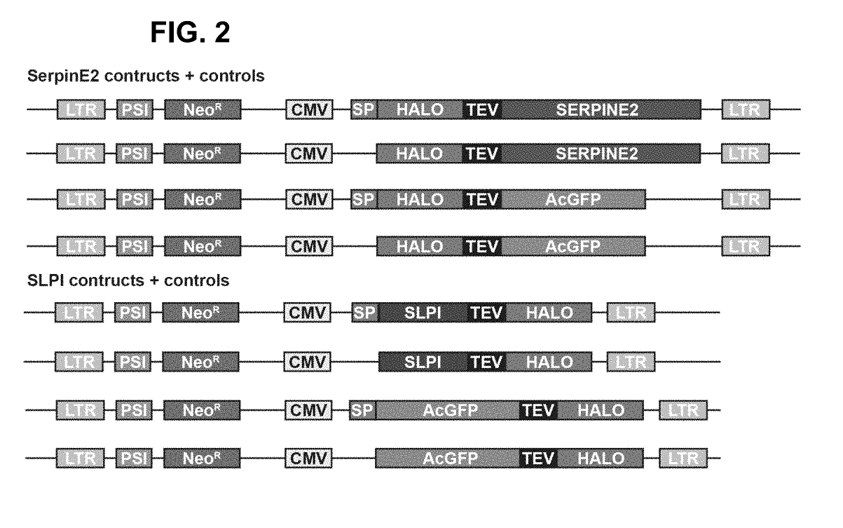

Both SerpinE2 and SLPI are secreted proteins but whether their secretion is necessary for their effects on VM is unknown. To ascertain the importance of intra- vs extracellular localization of SerpinE2 and SLPI, we perform knockdown add-back experiments in which we suppress SerpinE2 or SLPI in 4T1-T^{VM} cells using shRNAs directed against the 3′ UTR followed by re-expression of HALO-tagged coding sequence constructs (FIG. 2) in which the signal peptide (a necessary signal for secretion) is either present or absent⁸. These engineered cell lines then are tested for their ability to form VM tubes in vitro in the Matrigel assay and their ability to form VM channels and metastases in vivo by transplantation into the mammary fat-pad of Balb/C mice.

B. Identification of SerpinE2- and SLPI-Target Proteins via Affinity Purification Coupled with Mass-Spectrometry.

Using HALO-tagged versions of SerpinE2 and SLPI (FIG. 2) generated and characterized as described in A above, we affinity purify their interacting proteins. The HALO tag is a mutated form of a bacterial haloalkane dehalogenase that forms a covalent linkage with chloroalkane ligands⁹. Fusing a HALO tag to a protein of interest therefore facilitates covalent capture of the protein of interest along with its interacting proteins by reacting with chloroalkane-functionalized sepharose beads. SerpinE2 and SLPI are both serine protease inhibitors but fall into distinct sub-classes based on their mechanisms of action. SLPI is a member of the canonical class of protease inhibitors that mimic an ideal substrate of their target protease¹⁰, whereas the Serpin family use an inhibition-by-deformation mechanism that involves a covalent Serpin:protease intermediate that drastically alters the conformation of the target protease¹⁰. Ordinarily the Serpin:protease complex is degraded by the proteasome but in isolation in vitro this complex is incredibly stable with a half-life of over one year¹¹. To identify SerpinE2 target proteases we take advantage of this covalent intermediate, which we stabilize by inhibiting the proteasome, and the covalent HALO-tag:resin interaction to perform very high stringency washes and identify high-confidence interacting proteins. We adopt a similar strategy to identify SLPI interactors but due to a lack of covalent SLPI:protease intermediate it are necessary to perform less stringent washes.

For all pull-down experiments, we compare proteins recovered with SerpinE2 or SLPI to those pulled down with acGFP as a negative control in addition to comparisons between constructs +/− signal peptide. Following pull-downs using HALO-link resin, proteins are liberated from the beads by TEV cleavage between the HALO tag and protein of interest followed by digestion of proteins into peptides and identification by mass-spectrometry (in collaboration with the proteomics core at Rockefeller University). These analyses identify SerpinE2- and SLPI-targets that then are functionally tested for their role in VM by shRNA knockdowns followed by VM tube assays in vitro and VM/metastasis assays in vivo.

If successful, the experiments described herein define where and how SerpinE2 and SLPI drive VM and define their target proteins whose downstream effects are further explored in the future. It is likely that the relevant targets of SerpinE2 and SLPI are extra-cellular proteases, if this is the case we explore the effects of these proteases on the proteome via approaches such as PROTOMAP¹² in which proteolytic fragments are mapped by coupling gel-based size selection with protein ID by mass-spectrometry.

### EXAMPLE 3

### Mechanistic Dissection of an Ire1-Restrained Secretory Program that is Essential for VM

To understand the mechanistic details of how IRE1 restrains VM, we use CLIP to define direct IRE1-target mRNAs, motifs, and nucleotide-resolution binding sites. We then assay the newly defined target genes for their impact on VM and metastasis and determine how these effects are exerted.

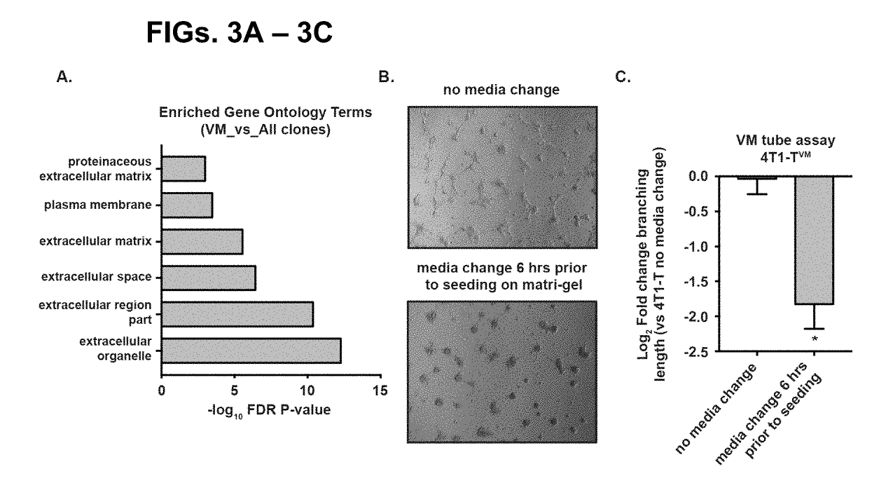

Our preliminary data indicate that VM clones up-regulate the expression of many genes encoding secreted extra-cellular factors (FIG. 3A) such as ECM (extra-cellular matrix) and ECM regulatory proteins. By performing experiments where we simply exchange the medium on VM clones prior to replating the cells on Matrigel, thus depriving the cells of high concentrations of cell-derived secreted factors, we have shown that VM clones broadly require secretion of extra-cellular factors to maintain their ability to form VM tubes in vitro (FIG. 3B-C). Given this striking result and the documented ECM-richness of VM tubular structures formed in vivo^{4,5} we hypothesized that regulators of the secretory pathway may influence the ability of tumor cells to perform VM and thus metastasis.

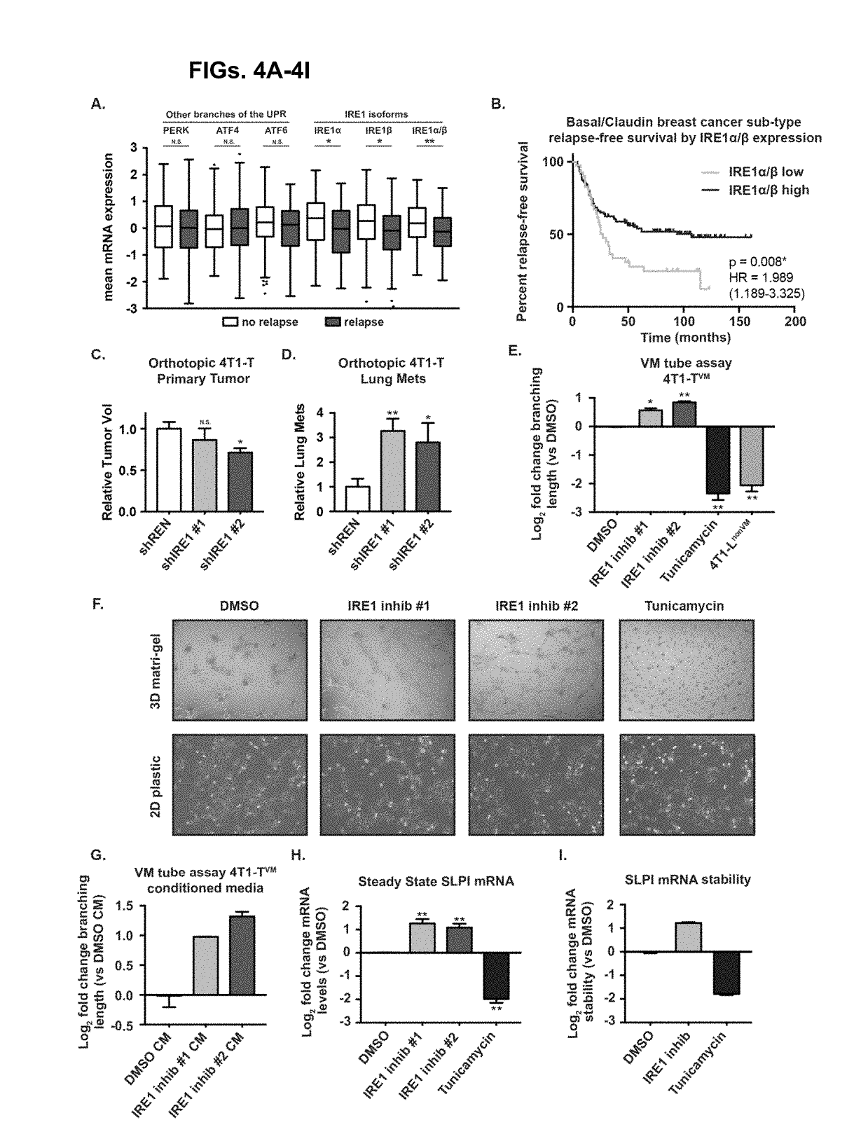

To prioritize candidates with clinical relevance we looked at the mRNA expression levels of critical regulators of secretion in patients with aggressive basal/claudin-low breast cancer that either did or did not relapse with metastatic disease¹³. We focused our analysis on regulators of the endoplasmic reticulum (ER) stress/unfolded protein response (UPR) as highly secretory cells have an increased load on the ER as the site of synthesis of most secreted proteins. As such, the UPR plays a critical role in adaptive responses that help negate increased traffic through the ER by a variety of mechanisms¹⁴. Analysis of the three branches of the UPR: PERK, ATF4 (a critical downstream target of the PERK pathway), ATF6 and IRE1, revealed a clear and selective downregulation of the IRE1 branch of the UPR in patients with metastatic relapse (FIG. 4A). Both the IRE1α and the IRE1β isoforms were suppressed and when considering their aggregate mean expression, the suppression was even more significant (FIG. 4A, p=0.0026). Furthermore, segregation of patients based on mean IRE1α/β expression demonstrated that patients in the bottom quartile had significantly shorter relapse-free survival than patients in the top 75% (FIG. 4B) suggesting that suppression of the IRE1 pathway in breast cancer patients favors metastasis and poor prognosis.

To determine whether IRE1 plays a causal role in metastasis, we transduced 4T1-T^{VM} cells with two different shRNAs targeting IRE1 and transplanted them orthotopically into the mammary fat-pad of syngeneic Balb/C mice. Subsequently, we measured primary tumor volume and harvested the lungs to enumerate metastases by IHC (immunohisto-chemistry) staining for mCherry contained within the shRNA vector. Knockdown of IRE1 had relatively minor effects on primary tumor burden (FIG. 4C) but increased the number of metastatic lung nodules 3B4 fold with two independent shRNAs (FIG. 4D).

Considered together, these data strongly suggest that suppression of IRE1 favors metastasis in both human patients and mouse models. To ascertain whether regulation of VM underlies these effects of IRE1 on metastasis we turned to the in vitro VM tube assay. Inhibition of IRE1 with two independent small molecule inhibitors of its RNAse activity enhanced VM tubulogenesis while activation of IRE1 with Tunicamycin suppressed VM (FIGS. 4E and F). Plating the remaining treated cells in 2D ruled out any demonstrable effects on cell viability that might account for suppression of VM by Tunicamycin (FIG. 4F).

IRE1 is a ER trans-membrane kinase/ribonuclease that responds to unfolded protein accumulation in the ER by two main mechanisms: (1) it cleaves a retained intron within the mature XBP1 mRNA which results in a frame shift and an active transcription factor that drives the expression of chaperones and other genes which increase the folding capacity of the ER¹⁵ (2) it directly cleaves mRNAs encoding secreted and membrane proteins leaving free 5′ and 3′ ends that are substrates for degradation in a process termed Regulated IRE1-Dependent mRNA Decay (or RIDD)¹⁵⁻¹⁷. Given the importance of secretion for VM (FIGS. 3A-3C) and the role that RIDD plays in restraining secretion we hypothesized that the IRE1 effects we observed on VM may are mediated by secreted factors.

To test this hypothesis, we acutely (4 hours) treated 4T1-T^{VM} cells with IRE1 inhibitors, followed by extensive washes with PBS (to remove remaining drug) and replacement with fresh medium. We then allowed the treated cells to condition the media for 24 hours, followed by filtration (to remove cells) and addition to naïve 4T1-T^{VM} cells for 24 hours followed by replating onto Matrigel for VM tube assays. As shown in FIG. 4G conditioned medium (CM) from IRE1 inhibitor-treated cells enhanced VM (FIGS. 4E and F), suggesting that IRE1 restrains the expression of secreted VM drivers. Moreover, IRE1 inhibition increased and activation decreased SLPI mRNA levels (FIG. 4H) and mRNA stability (FIG. 4I) suggesting that SLPI mRNA may are at least one target of RIDD relevant to its effects on VM.

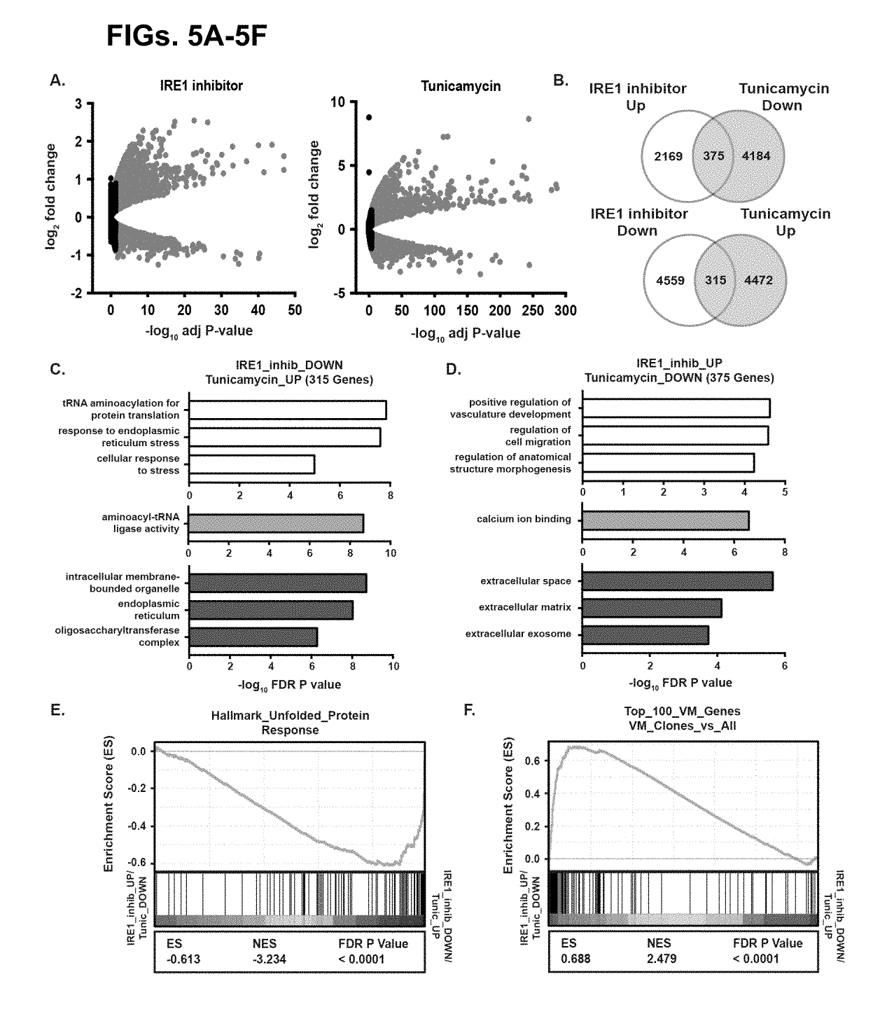

To ascertain the effects of IRE1 inhibition or activation on the transcriptome more broadly we performed RNA-Seq on polyA+RNA extracted from 4T1-TM^{VM} cells treated with an IRE1 inhibitor or Tunicamycin looking for gene expression changes that were mirrored in the two conditions, since they have opposing effects of IRE1 activity and VM (FIG. 5A-B) Gene Ontology (GO) analysis of genes significantly (DESeq corrected p value<0.05) up-regulated by Tunicamycin/IRE1 activation and significantly down-regulated by IRE1 inhibition showed enrichment of terms related to ER stress as expected (FIG. 5C). Strikingly however, genes that were significantly down-regulated by Tunicamycin/IRE1 activation and significantly up-regulated by IRE1 inhibition were enriched for regulators of vasculature development and secreted ECM/ECM-affiliated genes (FIG. 5D). Moreover, gene set enrichment analysis (GSEA)¹⁸ of a rank ordered list of the mean of the log² fold change in IRE1 inhibitor treated cells and the inverse log² fold change in Tunicamycin treated cells revealed that a VM signature, derived from our analysis of VM clones vs all other clones, was globally up-regulated by IRE1 inhibition (FIG. 5F). Together these data suggest that tumor cell IRE1 restrains a broad program of mRNAs encoding secreted ECM proteins and regulators of vasculature development that are necessary for vascular mimicry and metastasis (FIGS. 4A-5F).

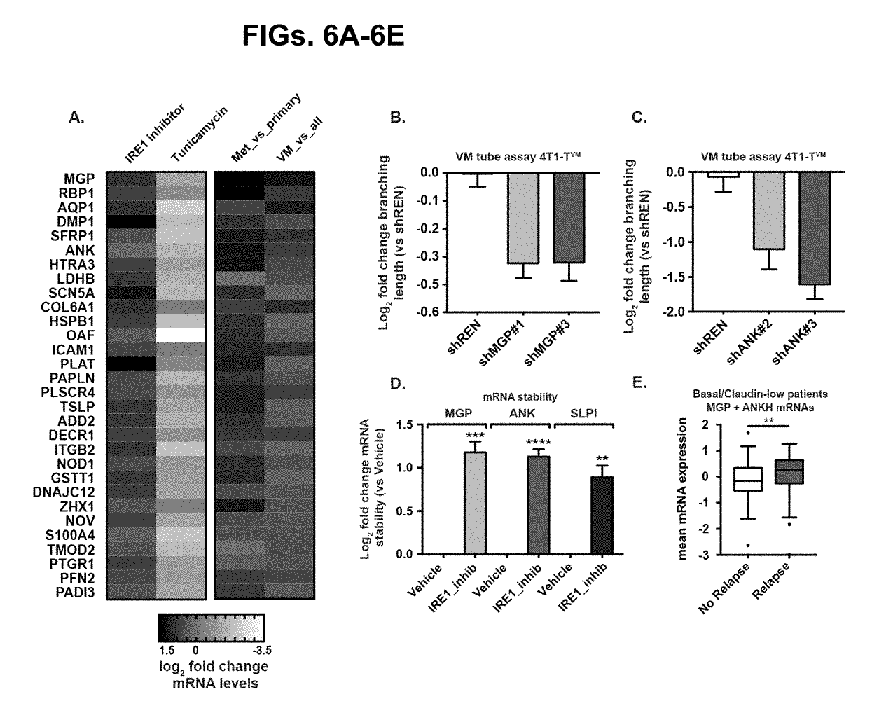

To begin to identify functionally important targets of IRE1 that contribute to it's suppression of VM we first ranked the genes that significantly increased with IRE1 inhibition and significantly decreased with Tunicamycin treatment by their loge fold change in VM clones (vs all other clones) and lung metastases (vs the primary tumor) (FIG. 6A). In continuing work we are knocking down each of these genes with RNAi and assaying their effects on VM tube formation of the ˜12 genes tested so far inhibition of 2 individual genes (MGP and ANK) results in demonstrable suppression of tube formation (FIGS. 6B and 6C). These mRNAs show significant increases in mRNA stability upon IRE1 inhibition (FIG. 6D), as measured by Actinomycin D run-off experiments, suggesting that they are targets of RIDD. We are currently in the process of measuring mRNA stability changes upon IRE1 inhibition transcriptome-wide using RNA-Seq. These data combined with the IRE1-CLIP (outlined below) define the landscape of functional, direct, IRE1 target mRNAs of importance for VM regulation. Moreover, the levels of MGP and ANKH (the human ortholog of mouse ANK) are up-regulated in basal/Claudin-low breast patients that relapse with metastatic disease consistent with a role in VM-mediated metastasis (FIG. 6E).

Our preliminary RNA-Seq data suggest that IRE1 inhibition up-regulates and stabilizes mRNAs encoding secreted proteins and regulators of vasculature development. The up-regulation and stabilization of mRNAs encoding secreted proteins is consistent with the known functions RIDD¹⁵⁻¹⁷. However, whether those specific RNAs, especially those RNAs encoding regulators of vasculature development, are direct targets of IRE1 binding and cleavage is unknown. Moreover, a comprehensive catalogue of IRE1 client RNAs is currently lacking.

To identify direct IRE1-target RNAs, motifs, and cleavage sites to be prioritized for further functional analysis, we perform IRE1-CrossLink Immuno-Precipitation (CLIP)¹⁹. IRE1-CLIP enables RNA-protein interactions to be probed unbiasedly on a transcriptome-wide scale. This technique involves ultra violet (UV) light-induced crosslinking to covalently link RNA-protein complexes in situ. Cells are then lysed and subjected to partial RNAse digestion to yield short RNA fragments directly bound by the protein of interest. Subsequently, the protein of interest is retrieved by immune-precipitation, linkers ligated and a cDNA library is generated and subjected to high-throughput sequencing. Since one or two amino acids can remain attached to the RNA, these can induce errors in reverse transcription (RT) that are informative for defining exact RNA-protein interaction sites using Cross-linking Induced Mutation Sites (CIMS) analysis^{19,20}. To facilitate IRE1-CLIP we employ a strategy using CRISPR/Cas9-based genome editing to epitope tag IRE1 at its endogenous locus in 4T1-T^{VM} cells.

We use a single stranded repair template encoding an HA tag and regions of homology to IRE1 to repair a CRISPR/Cas9-induced double strand break near the N-terminus of IRE1. Desired clones are identified by PCR followed by digestion with a restriction enzyme that is silently encoded within the repair template. After UV crosslinking, anti-HA IPs are performed on control/untagged 4T1-T^{VM} cells and N-terminal HA-IRE1 4T1-T^{VM} cells to control for background from the HA-IP in the absence of any confounding underlying changes in gene expression. Bound crosslinked RNAs are partially RNAse digested, linkers are ligated and then converted to cDNA libraries for high throughput sequencing.

Peaks of binding are defined by standard peak height analysis and specific interaction sites defined by CIMS analysis. Over-represented motifs are identified using the MEME suite²¹ and validated by fusing sequences to reporter constructs resulting in refinement of the IRE1 cleavage motif in addition to discovery of potential cis regulatory proteins of IRE1-target RNAs. Together these experiments produce the first transcriptome-wide map of IRE1:RNA interactions, refine cleavage-site motifs, and by examining RNAs whose levels change upon IRE1 perturbation in our RNA-Seq dataset define rules that explain the impact of IRE1 binding on RNA metabolism.

To determine the functional impact of novel IRE1-target RNAs on VM and metastasis, the direct IRE1-target RNAs identified above are analyzed for changes in abundance in the IRE1 inhibition/activation RNA-Seq dataset. These high-confidence targets then are assayed for changes in mRNA stability upon IRE1 inhibition/activation by performing Actinomycin D run-off experiments, as done for SLPI mRNA (FIG. 4I) and MGP/ANK (FIG. 6D), to determine whether they are RIDD substrates. This results in a relatively short list of high-confidence RIDD substrate RNAs that then are assayed for their functional effects on VM tube formation and metastasis. We generate shRNAs targeting the top 20 RIDD substrates and test the effect of individual knockdowns on the ability of 4T1-T^{VM} cells to form tubular structures in Matrigel. We expect that individual knockdowns demonstrably reduce tube formation, but may fail to reach the extent of reduction observed with Tunicamycin. Of those targets that effect tube formation individually, we then knockdown pairs of target RNAs using next-generation vectors, developed in our lab, harboring multiple shRNAs.

Candidates that exhibit strong effects on tube formation in vitro are assayed for their effects on VM in vivo by transplanting knockdown cells into the mammary fat-pad of Balb/C mice and performing immunohistochemistry (IHC)BVM staining of primary tumors looking for periodic acid Schiff (PAS)-positive, mCherry-positive, CD31-negative channels as we have done previously (FIG. 1EBF). This staining strategy seeks to identify ECM-rich channels (staining positively for PAS), that are of tumor origin (mCherry positive, contained within the shRNA vector transduced into the cells ex vivo) and not endothelial cells (CD31 negative, a marker of normal endothelial cells).

Primary tumor burden and pulmonary metastases are measured to assess the effects of IRE1/VM genes on tumor growth and spread as we have done for IRE1, SerpinE2 and SLPI (FIG. 1D and FIGS. 4C to 4D). Those targets whose loss impacts VM and metastasis are analyzed for their clinical relevance by examining their mRNA levels in breast cancer patient datasets, specifically looking for their association with relapse-free survival as we have done for IRE1 (FIGS. 4A-4B) and MGP/ANKH (FIG. 6E).

These experiments define a core set of direct IRE1/RIDD substrate RNAs that mediate its effect on VM which are explored for their relation to patient survival for prognostication. In future experiments, we delve into the detailed mechanisms by which these targets exert strategies to those employed for SerpinE2 and SLPI.

### EXAMPLE 4

### The FOXC2-Driven Epithelial-to-Endothelial (EET) Transition, and its Role in VM

We probe the importance of endothelial gene expression programs induced by FOXC2 and their role in VM and metastasis. We use FOXC2 CHIP-Seq in conjunction with RNA-Seq in FOXC2 loss-and gain-of-function systems to define direct targets and explore routes of FOXC2-mediated endothelial differentiation with single cell RNA-Seq. We also perform an RNAi screen of “druggable” genes to identify regulators of FOXC2 function.

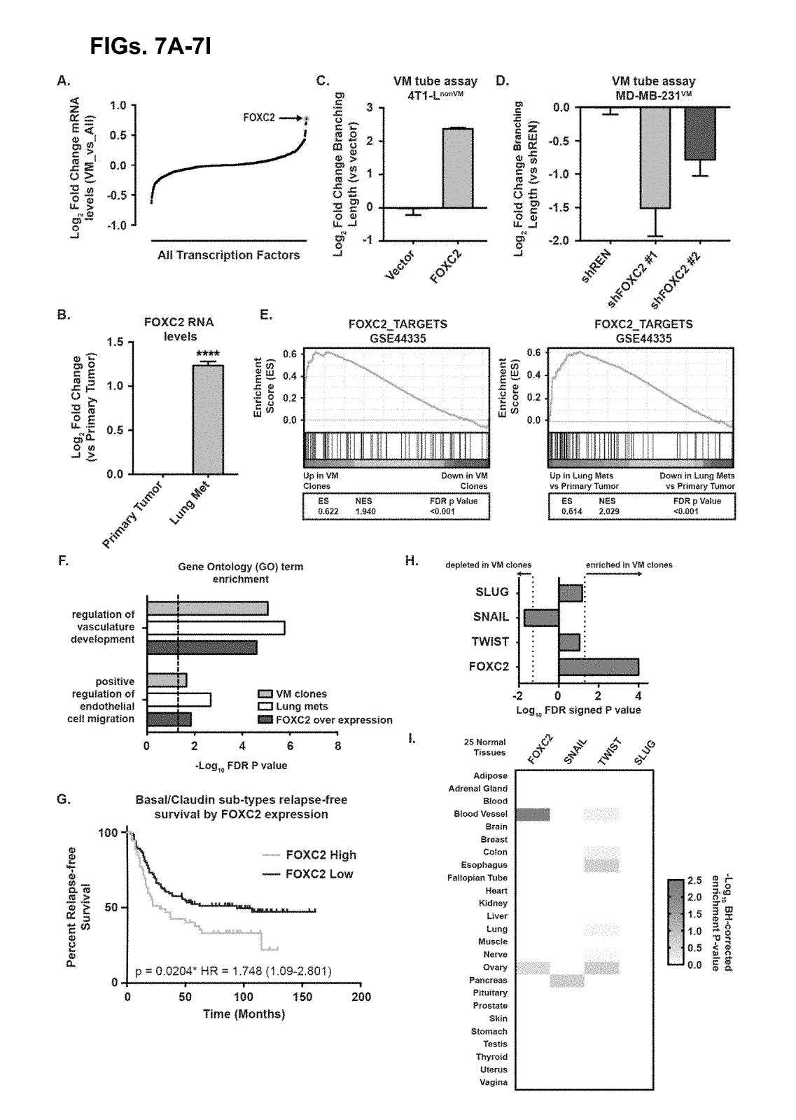

We reasoned that, like many phenotypic transitions, the acquisition of endothelial-like properties by VM tumor cells may are driven by a master transcription factor (TF). Therefore, we ranked all TFs by their change in mRNA expression between VM clones and all other clones and found that FOXC2 and one of its important target genes MEF2C were the 2nd and 3rd most up-regulated TFs respectively, in VM cells (FIG. 7A). FOXC2 mRNA levels were also significantly elevated in cell lines derived from lung metastases relative to the primary tumor (FIG. 7B) highlighting a potential role in metastasis. FOXC2 was of interest because it is critically important in normal endothelial development. Mice lacking both copies die pre- and perinatally with profound cardiovascular defects²⁴, and in collaboration with ETS transcription factors, FOXC2 can specify gene expression to the endothelium²⁵. Moreover, direct reprogramming of fibroblasts into endothelial-like cells requires up-regulation of FOXC226, suggesting a critical role in trans-differentiation of non-endothelial cell types to endothelium. Given the importance of FOXC2 in vascular development, endothelial gene expression and trans-differentiation we hypothesized that FOXC2 up-regulation would are sufficient to drive non-VM cells towards a VM/endothelial-like state. Indeed, enforced expression of murine FOXC2 in one of the most VM-deficient clones, 4T1-L^{nonVM}, was sufficient to induce tube-forming potential (FIG. 7C). Conversely, depletion of FOXC2 in VM-competent human breast cancer cells (MDA-MB-231^{VM}), suppressed tubulogenesis (FIG. 7D). To determine whether a broader FOXC2-driven transcriptional program was operative in VM cells we utilized a publicly available gene expression dataset, of Human Mammary Epithelial (HMLE) cells over-expressing FOXC2 (GSE44335), to define a set of FOXC2-target genes. Gene set enrichment analysis (GSEA)¹⁸ demonstrated that FOXC2-target genes were globally up-regulated in VM clones relative to all other clones (FIG. 7E left panel) and in lung metastases relative to primary tumors (FIG. 7E right panel). Gene Ontology (GO) analysis revealed a significant up-regulation of genes involved in vasculature development in epithelial cells over-expressing FOXC2 to a similar extent as seen in VM clones or 4T1-derived lung metastases (FIG. 7F). Analysis of breast cancer patients revealed significantly shorter relapse-free survival times in basal/claudin-low patients with high FOXC2 expression (FIG. 7G). Together these data indicate that FOXC2 is both necessary and sufficient to establish a VM state de novo, suggesting that it may are a master regulator of vascular mimicry and metastasis by inducing an epithelial-to-endothelial transition (EET).

FOXC2 has been previously shown to promote metastasis in 4T1 breast tumors and has been implicated in another form of trans-differentiation, the epithelial-to-mesenchymal transition(EMT)²⁷. EMT involves the loss of epithelial characteristics and gain of mesenchymal characteristics including increased migratory capacity and loss of cell-cell contacts and is thought to mediate metastasis through these effects²⁸. However, from our preliminary data we would hypothesize that FOXC2 promotes metastasis through VM raising the critical question of whether FOXC2-driven metastasis is mediated via EMT, EET or both. To begin to address this question we first asked whether VM clones have undergone an EMT at the gene expression level by analyzing the enrichment of target-genes of bona fide EMT transcription factors (TFs) in VM clones using GSEA and signatures derived from gene expression data of HMLE cells overexpressing the EMT TFs, TWIST, SNAIL, or SLUG (GSE43495). Only FOXC2 targets were significantly enriched in VM clones (FIG. 7H), suggesting that VM clones don't manifest a “typical” EMT signature at the gene expression level. Moreover, Tissue-specific Expression Analysis (TSEA) (http://genetics.wustl.edu/jdlab/tsea/), where tissue-specific genes across 25 normal tissue types are interrogated for enrichment in a gene list, demonstrated that FOXC2-target genes (defined in normal epithelial cells) but not EMT TF-target genes (defined in the same epithelial cell type) encompass genes that are normally specific to blood vessels (FIG. 7I). These data strongly argue that FOXC2 induction drives expression of a set of vascular genes in non-endothelial cells and that this is not a common feature of the EMT but do not rule out the possibility that an EMT is a required intermediate of an EET. In fact, the EMT has been linked to the acquisition of “stem cell”-like characteristics²⁹ and as such may act as a transitionary step between an epithelial and endothelial state.

To define direct targets of FOXC2, we use CHIP-Seq /RNA-Seq to understand the EET. A key question is whether FOXC2 mediates a direct conversion of epithelial tumor cells to an endothelial-like state or whether it requires an intermediate mesenchymal state and ultimately what is the relative importance of FOXC2-driven EMT vs EET for metastasis. To answer this question, we first require a high-confidence, high-resolution picture of FOXC2-mediated transcriptional control in VM cells to identify direct target-genes of FOXC2 that encode endothelial or mesenchymal genes allowing the delineation of the EMT and EET effects of FOXC2 over-expression. To identify direct FOXC2 target-genes we adopt an analogous strategy to that employed for IRE1 in Example 3, in that we endogenously tag the FOXC2 locus and perform CHIP-Seq (Chromatin-Immuno-Precipitation). We couple the CHIP-Seq with RNA-Seq to determine gene expression changes upon FOXC2 over-expression in non-VM 4T1 clones and FOXC2 knockdown 4T1-T^{VM} cells.

Since, FOXC2 has been shown to function cooperatively with other TFs, e.g., the ETS family, in defining the expression of endothelial genes²⁵, we perform motif enrichment analysis of regions surrounding FOXC2-binding peaks in both endothelial and mesenchymal genes. We utilize these new data along with our existing data from HMLE cells over-expressing FOXC2 and MDA-MB-231 cells with FOXC2 knockdown to define high-confidence endothelial and mesenchymal genes controlled by FOXC2. We then perform loss-of-function experiments with endothelial or mesenchymal FOXC2-targets and assay their effects on VM tube formation and EMT markers to determine whether (a) loss of EMT drivers influences VM, (b) loss of EET drivers alters VM while sparing the mesenchymal state, and/or (c) whether perturbation of specific FOXC2 co-factors allows separation of VM/EET and EMT functions of FOXC2.

Similarly, we ascertain whether FOXC2 over-expression is still able to induce VM in cells where it cannot induce and EMT such as those with Zeb1/2 knockdown. Having ascertained genes whose disruption specifically impede the EMT or EET functions of FOXC2, we then assay their effects on metastasis in vivo as we have for IRE1, SerpinE2 and SLPI (FIGS. 1A-1G and 4A-4I). Together these data define the mechanisms by which epithelial tumor cells transition to an endothelial-like state and the relative importance of the EMT and EET to this process and to metastasis.

To define the pathways of naturally arising EET differentiation, we employ single-cell RNA-Seq. Comparison of VM clones with non-VM clones from a collection of 4T1-derived cell lines has yielded insights into the molecular underpinnings of vascular mimicry. However, based on the original large-scale analysis of ˜400 4T1 sub-clones⁷, we suspect that the 23 sub-clone system only captures a subset of possible cell states within 4T1 parental cells and breast tumors more broadly. To understand differentiation state transitions in more detail, such as routes of differentiation via intermediate and precursor cell types, single-cell analysis is likely to be much more informative.

We deploy scRNA-Seq to identify VM cells within parental 4T1 cells in culture and parental 4T1-derived tumors and use this data to cluster cells based on a spectrum of similarity in gene expression with VM cells. The single cell platform that we use is a commercial platform offered by 10× genomics³⁰, based on a modified version of the “Drop-seq” protocol³¹, which involves the encapsulation of a single cell within a nanoliter lipid droplet containing a single micro-particle bead coated with barcoded primers. For analysis, we deploy a computational framework called Seurat (http://satijalab.org/seurat/), which enables normalization, dimensionality reduction, clustering, and data visualization all within a package for the R statistical computing environment. We first use our FOXC2 CHIP-Seq/RNA-Seq profiles generated in Example 4 to define an EET gene signature and then after performing scRNA-Seq on 4T1 cells and 4T1-derived tumors we use this signature to identify high confidence VM cells. Then using the gene expression of those cells, we cluster all cells within the population based on their similarity to VM cells. This analysis facilitates the identification of precursors to VM cells and allow us to draw a roadmap of EET differentiation. Through iterative rounds of analysis, we derive a minimal signature that can identify VM cells in single cell data that can are deployed in the future to determine the VM content of patient tumors. Although our primary goal is to define discrete stages of EET differentiation, these single cell datasets are invaluable to understanding tumor heterogeneity and phenotypically distinct sub-populations within tumors that may influence disease progression or response to therapy.

To identify chemically accessible regulators of FOXC2/VM, we use RNAi screening. Pharmacological targeting of FOXC2 is an attractive strategy for prevention of secondary metastases in addition to a potential avenue for circumventing resistance to anti-angiogenic therapy. However, transcription factors are notoriously difficult to target directly with small molecules. Fortunately, many TFs, including FOXC232, are targets of kinases which modulate their stability and/or require specific co-factors or chromatin environments for their function. Thus, targeting of these regulators is an alternative strategy to modulate key oncogenic/metastatic TFs³³. For example, FOXC2 is a target of p38 map kinase which promotes FOXC2 protein stability such that inhibition of p38 leads to a decrease in FOXC2 protein levels due to increased turnover³². To identify additional modulators of FOXC2 with a focus on its VM-promoting properties, we perform an RNAi screen against the “druggable” genome looking for targets whose suppression leads to loss of expression of an endogenously-encoded reporter of FOXC2 activity.

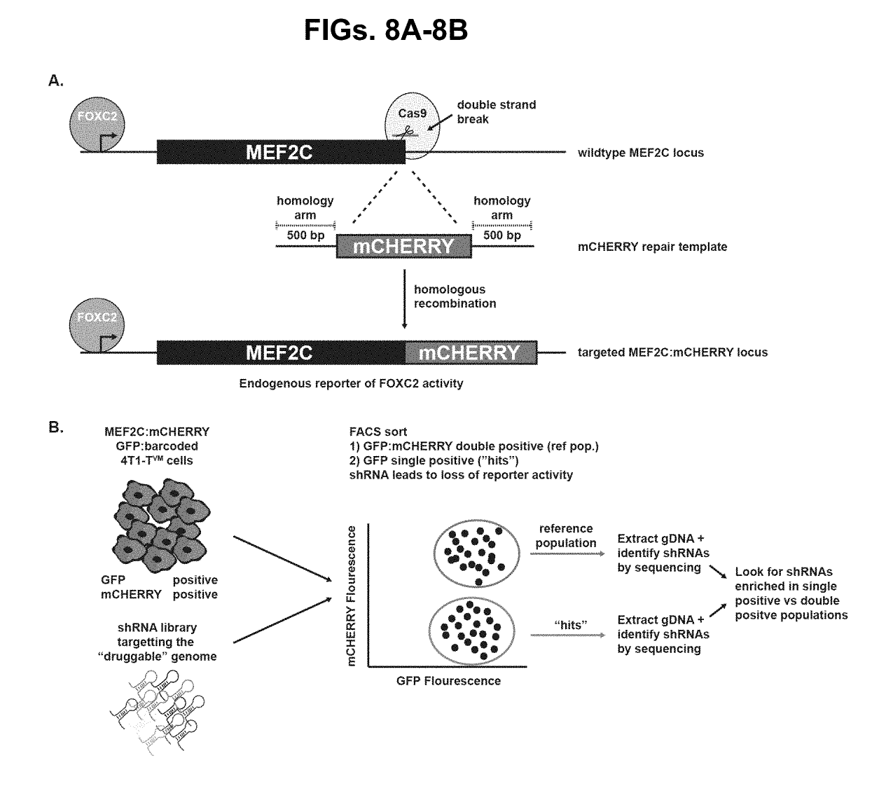

We employ a library that we designed and is now commercially available in arrayed format through transomics. Our reporter system is based on an important FOXC2-target gene in endothelium, MEF2C³⁴, which is also up-regulated in VM clones. We tag the endogenous MEF2C locus in 4T1-T^{VM} cells with mCherry and use it as a fluorescence read-out of FOXC2 activity for our screen (FIGS. 8A-8B). Since the barcode vector (that was used to generate the 4T1-T^{VM} cells) contains GFP, we can sort the cells into two populations that represent our background population (positive for both GFP and mCherry) and our “hits” (GFP-high and mCherry-low). FOXC2- and mCherry-targeting shRNAs serve as positive controls (enriched in GFP-high/mCherry-low cells) and shRNAs targeting Renilla luciferase serve as negative controls (enriched in GFP+/mCherry+cells). We identify “hits” as those that have ≥2 hairpins targeting the same gene enriched in the GFP-high/mCherry low population relative to the GFP+/mCherry+population with an enrichment >2 standard deviations from the negative control hairpins. Genes identified as “hits” in the screen are validated by qRT-PCR of endogenous MEF2C and other important FOXC2-target genes in 4T1-T^{VM} cells expressing individual “hit” hairpins; and also by VM tube assay of individual knockdowns. In cases where either approved drugs or tool compounds are available to individual hits, we test these in the same assays.

Together these data identify routes of endothelial differentiation, key regulatory networks that enable FOXC2 to drive VM and metastasis, and provide a foundation for assessing the feasibility of targeting the FOXC2-driven EET pharmacologically. These data also potentially provide a framework for exploring the role of other differentiation state transitions in cancer.

### EXAMPLE 5

### The Role of Vm in Response to Anti-Angiogenic Therapy in Mouse Models and Patient-Derived Xenografts (PDXS)

Based on the information provided in Examples 1-4, we ascertain the importance of VM in determining response to anti-angiogenic therapy. Genetic and pharmacological manipulation of VM-competent tumor models is combined with anti-angiogenics, potentially leading to preclinical validation of targeting VM as a combination therapeutic strategy.

We us our VM regulators defined above to ascertain the relationship between VM and response to anti-angiogenic therapy and the effects of genetic and pharmacological manipulation of VM on response of 4T1-derived breast tumors and breast cancer patient-derived xenografts (PDXs) to anti-angiogenic therapy. Inhibitors of angiogenesis, such as the VEGF-blocking antibody Avastin®/bevacizumab, have shown disappointing results in clinical trials displaying variable responses in multiple tumor types especially breast cancer (https://www.cancer.gov/about-cancer/treatment/drugs/fda-bevacizumab). Various resistance mechanisms have been postulated to underlie failure of anti-angiogenic therapy, including VM; however compelling clinically-relevant evidence for VM-mediated resistance is lacking.

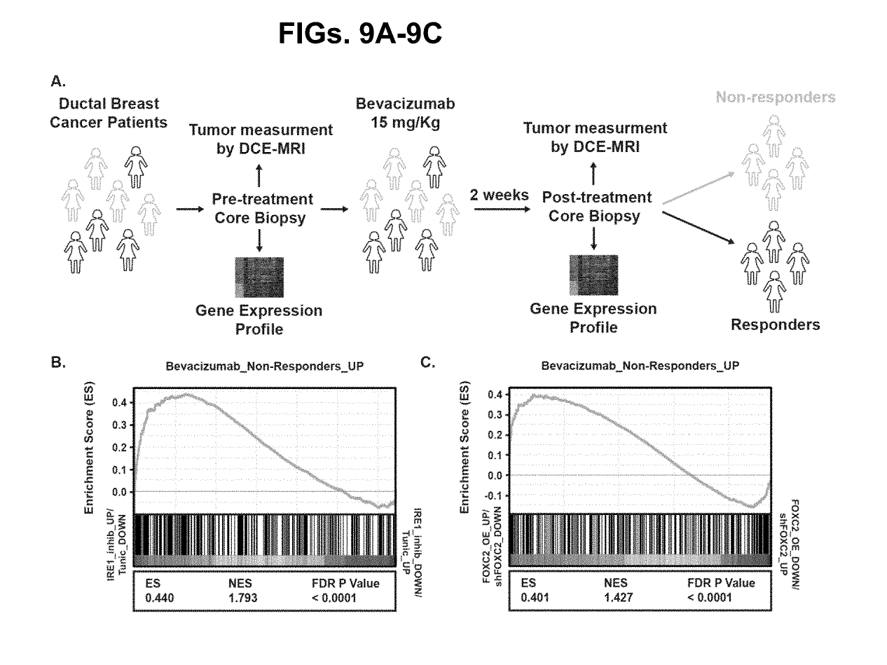

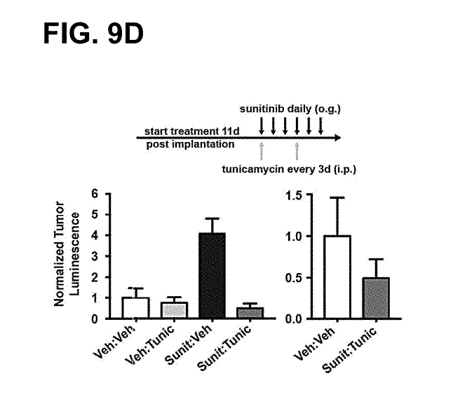

We address the role of VM in clinical resistance to anti-angiogenic therapy using a recently published study³⁸ in which previously untreated ductal breast cancer patients received neo-adjuvant, single agent, bevacizumab (Bev) for 2 weeks. Core biopsies were taken immediately prior and two weeks post-therapy, RNA extracted and subjected to gene expression profiling by microarray. The authors additionally measured tumor response to Bev via MRI imaging before and after therapy facilitating the correlation of gene expression changes with a quantitative measure of the anti-tumor activity of Bev (FIG. 9A)³⁸. The authors identified a set of genes whose levels were significantly elevated in patients who failed to respond.

To assess the role of VM as a resistance mechanism in this clinical cohort, we examined whether this Bev resistance gene signature was altered by perturbations that influence VM (FIGS. 9A-9C). Gene set enrichment analysis of the IRE1-regulated (FIG. 9B) or FOXC2-regulated (FIG. 9C) gene expression data sets that we have generated demonstrated that Bev resistance genes are globally up-regulated by IRE1 inhibition (FIG. 9B) or FOXC2 over-expression (FIG. 9C), perturbations that enhance VM, and globally down-regulated by IRE1 activation (with Tunicamycin) or FOXC2 knockdown, perturbations that restrain VM. To our knowledge these data provide the first clinically-relevant evidence that failure to respond to anti-angiogenic therapy in patients is associated with increased expression of VM-related genes. Thus, co-targeting of VM may enhance response to anti-angiogenic cancer therapies.

Additional preliminary data is shown in FIG. 9D suggesting that drug regimens that impede VM in combination with angiogenesis inhibition improve tumor responses to anti-angiogenic agents. That figure shows the results of treating 4T1-TVM-derived tumors with Vehicle, Tunicamycin alone, Sunitinib (anti-angiogenic kinase inhibitor) alone, or a combination of Tunicamycin and Sunitinib using regimens as indicated. Tumor volumes were measured by bioluminescence prior to and after cessation of therapy. Bioluminescent tumor volumes normalized to pre-treatment and vehicle treated tumors at day 6 after initiation of therapy are shown. Data suggest that 4T1-TVM tumors are resistant to Sunitinib and are sensitized to Sunitinib by inhibiting VM in combination by using Tunicamycin as an IRE1 activator.

In additional studies, we want to determine the effect of anti-angiogenic therapy on VM-proficient and VM-deficient 4T1 sub-clones and address the role of VM as a mediator of resistance to anti-angiogenic therapy. First, we ascertain the relative effects of anti-VEGF antibodies that recognize both the human and murine VEGF, i.e., B20B4.1.1 (Genetech³⁹), or Sunitinib (Pfizer, a small molecule multi-angiogenic receptor inhibitor) on tumors generated from 4T1-derived sub-clones with known VM capabilities. Others have shown that parental 4T1 tumors demonstrate moderate responses to anti-VEGF antibodies or Sunitinib showing a ˜40% and ˜30% reduction in tumor volume, respectively, over a typical course of treatment⁴⁰. To determine whether these moderate effects are determined by the relative ability of sub-populations of tumor cells to perform VM, we derive tumors from pure populations of VM and non-VM clones. Once tumors have established, we determine the efficacy of anti-angiogenic therapy against these extreme VM scenarios. We expect that non-VM-derived tumors are sensitive and VM-derived tumors are resistant. In heterogeneous tumors, we expect that regions of high VM are selectively spared cell death induced by anti-angiogenic therapy. To test this hypothesis, we label the two VM clones with mCherry and pool them with the 21 remaining unlabeled 4T1-derived clones. We then look at markers of cell death across tumor sections in regions proximal and distal to highly VM vascularized regions before and after therapy.

We stratify breast cancer patient-derived xenografts (PDXs) into VM-high and VM-low based groups to determine VM's effect on anti-angiogenic therapeutic response. Syngeneic mouse models of cancer, such as the 4T1 model, are invaluable tools for studying cancer biology in a host with an intact immune system while facilitating perturbations to tumor cells. However, no one mouse model can fully capture inter-individual variability seen between breast cancer patients nor capture intra-tumor heterogeneity within a single patient in its entirety. As such a generalized approach leveraging the advantages of murine models for discovery coupled with heterogeneity-preserving patient-derived xenografts (PDXs) for validation and pre-clinical testing is a powerful strategy. Through a collaboration with Prof Carlos Caldas at Cancer Research UK Cambridge, we have access to an extensive collection of molecularly and clinically annotated breast cancer PDXs, which we use to study the clinical relevance of VM, the potential of pharmacologically targeting VM, and its role in response to anti-angiogenic therapy. Specifically, we have access to >100 PDX models derived from patients with different sub-types of breast cancer for which we have gene expression and clinical data⁴¹. We first determine the VM capacity of each of these models by performing CD31/PAS immuno-histochemistry (IHC) staining on archived tissue sections from the PDX tumors looking for CD31 negative (a marker of normal endothelial cells) PAS positive (Periodic Acid Schiff Stain, a marker of basement membrane) channels. Our VM-score are the percentage of all vessels that demonstrate VM pathology as defined by CD31/PAS. We can also culture these PDX models short term to evaluate tube forming capacity. Having defined VM-high and VM-low PDX models we then interrogate existing gene expression data from these models for expression of our EET/FOXC2 and IRE1 genes defined in Examples 3 and 4 with the expectation that these genes would correlate with VM score, either in bulk samples or in single cell datasets.

We perform RNAi knockdowns of key VM mediators identified in Examples 1-4 in primary patient-derived cell lines and perform VM Matrigel assays in cell culture. Having thoroughly established the VM capabilities of individual PDX models, we then ascertain the therapeutic efficacy of anti-angiogenic therapy in a subset of these PDX models (2-3 patients representative of each VM-high,-med and-low states) using anti-VEGF antibodies that recognize both the human and murine VEGF (B20B4.1.1, Genetech³⁹) or Sunitinib. Drugs are administered to NSG sub-cutaneous-PDX-bearing mice following schedules that closely mirror clinical regimens. Tumor volumes are measured daily, normal blood vessel density and VM vessels are measured at experimental endpoints by CD31/PAS IHC. We expect that PDX tumors that are VM-high at baseline demonstrate an inferior response to anti-angiogenic therapy than VM-low tumors and that a sub-set of VM-med PDX tumors may induce VM upon treatment ultimately leading to treatment failure.

To determine the efficacy of combination anti-VM/anti-angiogenic therapy in 4T1 model and human PDX models, after having determined critical mediators and drivers of VM, we ascertain the effects of genetically suppressing VM on the response of murine breast tumors to anti-angiogenic therapy. To model targeting VM in tumors, we knockdown individual VM regulators, such as FOXC2, IRE1 and their target genes, or combinations thereof, in parental 4T1 cells and inject them orthotopically into the mammary fat pad of Balb/C mice. Once tumors have established, animals are treated with an anti-VEGF antibody (B20B4.1.1, Genetech³⁹) or Sunitinib using regimens that closely mimic those used clinically. Primary tumor burden, metastatic burden, and VM channels (by CD31/PAS) are measured as we have done previously (FIGS. 1 and 4) before and after therapy. These studies reveal the impact of manipulating VM on the ability of anti-angiogenic therapy to impact both primary and metastatic disease.

Having established a proof-of-principal for targeting VM genetically in the 4T1 model we next determine the effects of small molecule targeting of VM in 4T1 and PDX models on response to anti- angiogenic therapy. We test small molecules targeting promising candidates from our RNAi screen “hits” as well as rational combinations based on our current knowledge such as IRE1 activation by low-dose Tunicamycin (which can are achieved in vivo without apparent toxicity⁴²) or p38 inhibitors to target FOXC2 indirectly +/− anti-VEGF therapy. We first establish suitable dosing regimens and then administer anti-VM small molecules +/− anti-angiogenic therapy to 4T1-tumor or PDX-bearing mice and measure tumor volumes and VM (by CD31/PAS staining). Together these data establish the feasibility and relevance of targeting VM to improve anti-angiogenic therapy and identify useful tool or therapeutic compounds for further testing as components of combination therapy regimes.

Alternatively, we test the ability of indirect FOXC2 suppression, via p38 map kinase inhibition, as a pharmacological strategy to augment anti-angiogenic therapy in PDX models. Similarly, activation of IRE1, via low-dose Tunicamycin treatment, are tested for its ability to inhibit VM and improve response to anti-angiogenic therapy as this profoundly suppresses VM tubulogenesis and gene expression signatures of VM and bevacizumab resistance (FIGS. 4A-4I, 5A-5F and 9A-9C).

This data supports small molecule targeting of VM in combination with anti-angiogenic therapy. In another embodiment, we use a minimal VM-based gene signature as a bio-marker of response to anti-angiogenic therapy and as a means to identify sub-sets of patients for whom combination anti-VM/anti-angiogenic therapy is beneficial.

Each patent, patent application, and publication, including websites cited throughout the specification, and sequences identified in the specification or available publicly, is incorporated herein by reference. While the invention has been described with reference to particular embodiments, it is appreciated that modifications can be made without departing from the spirit of the invention. Such modifications are intended to fall within the scope of the appended claims.

### REFERENCES

1. Folkman, J. Tumor angiogenesis: therapeutic implications. N Engl J Med 285, 1182-1186 (1971).
2. Folkman, J., et al. Isolation of a tumor factor responsible for angiogenesis. J Exp Med 133, 275-288 (1971).
3. Sennino, B. & McDonald, D. M. Controlling escape from angiogenesis inhibitors. Nat Rev Cancer 12, 699-709 (2012).
4. Dome, B., et al. Alternative Vascularization Mechanisms in Cancer. Amer. J. Pathol. 170, 1-15 (2007).
5. Maniotis, A. J. et al. Vascular Channel Formation by Human Melanoma Cells in vivo and in vitro: Vasculogenic Mimicry. Amer. J. Pathol. 155, 739-752 (2010).
6. Yang, J. P. et al. Tumor vasculogenic mimicry predicts poor prognosis in cancer patients: a meta-analysis. Angiogenesis 1-10 (2016). doi:10.1007/s10456_016_9500_2
7. Wagenblast, E. et al. A model of breast cancer heterogeneity reveals vascular mimicry as a driver of metastasis. Nature 520, 358-362 (2015).
8. Devillers-Thiery, A., et al. Homology in amino terminal sequence of precursors to pancreatic secretory proteins. Proc. Natl. Acad. Sci. 72, 5016-5020 (1975).
9. Los, G. V. et al. HaloTag: a novel protein labeling technology for cell imaging and protein analysis. ACS Chem. Biol. 3, 373-382 (2008).
10. M Laskowski, A. I. K., Jr. Protein Inhibitors of Proteinases. 1-36 (2017).
11. Huntington, J. A., et al. Structure of a serpin-protease complex shows inhibition by deformation. Nature 407, 923-926 (2000).
12. Dix, M. M., et al. Global Mapping of the Topography and Magnitude of Proteolytic Events in Apoptosis. Cell 134, 679-691 (2008).
13. Harrell, J. C. et al. Genomic analysis identifies unique signatures predictive of brain, lung, and liver relapse. Breast Cancer Res Treat 132, 523-535 (2011).
14. Moore, K. A. & Hollien, J. The unfolded protein response in secretory cell function. Annu. Rev. Genet. 46, 165-183 (2012).
15. Maurel, M., et al. Getting RIDD of RNA: IRE1 in cell fate regulation. Trends in Biochemical Sciences 39, 245-254 (2014).
16. Hollien, J. & Weissman, J. S. Decay of endoplasmic reticulum-localized mRNAs during the unfolded protein response. Science 313, 104-107 (2006).
17. Hollien, J. et al. Regulated IRE1-dependent decay of messenger RNAs in mammalian cells. J Cell Biol 186, 323-331 (2009).
18. Subramanian, A. et al. Gene set enrichment analysis: a knowledge-based approach for interpreting genome wide expression profiles. Proc. Natl Acad Sci 102, 15545-15550 (2005).
19. Moore, M. J. et al. Mapping Argonaute and conventional RNA-binding protein interactions with RNA at single nucleotide resolution using HITS-CLIP and CIMS analysis. Nat Protoc 9, 263-293 (2014).
20. Weyn-Vanhentenryck, S. M. et al. HITS-CLIP and integrative modeling define the Rbfox splicing-regulatory network linked to brain development and autism. Cell Reports 6, 1139-1152 (2014).
21. Bailey, T. L. et al. MEME SUITE: tools for motif discovery and searching. Nucleic Acids Res 37, W202-8 (2009).
22. Chi, S. W., Hannon, G. J. & Darnell, R. B. An alternative mode of microRNA target recognition. Nat. Struct. Mol. Biol. 19, 321-327 (2012).
23. Karginov, F. V. & Hannon, G. J. Remodeling of Ago2-mRNA interactions upon cellular stress reflects miRNA complementarity and correlates with altered translation rates. Genes Dev. 27, 1624-1632 (2013).
24. Bryan. Essential roles of the winged helix transcription factor MFH-1 in aortic arch patterning and skeletogenesis. 1-12 (1997).
25. De Val, S. et al. Combinatorial Regulation of Endothelial Gene Expression by ETS and Forkhead Transcription Factors. Cell 135, 1053-1064 (2008).
26. Morita, R. et al. ETS transcription factor ETV2 directly converts human fibroblasts into functional endothelial cells. Proc. Natl. Acad. Sci. 112, 160-165 (2015).
27. Mani, S. A. et al. Mesenchyme Forkhead 1 (FOXC2) plays a key role in metastasis and is associated with aggressive basal-like breast cancers. Proc. Natl. Acad. Sci. 104, 10069-10074 (2007).
28. Tam, W. L. & Weinberg, R. A. The epigenetics of epithelial-mesenchymal plasticity in cancer. Nature Medicine 19, 1438-1449 (2013).
29. Mani, S. A. et al. The epithelial-mesenchymal transition generates cells with properties of stem cells. Cell 133, 704-715 (2008).
30. Zheng, G. X. Y. et al. Massively parallel digital transcriptional profiling of single cells. Nature Communications 8, 140-49 (2017).
31. Macosko, E. Z. et al. Highly Parallel Genome-wide Expression Profiling of Individual Cells Using Nanoliter Droplets. Cell 161, 1202-1214 (2015).
32. Werden, S. J. et al. Phosphorylation of serine 367 of FOXC2 by p38 regulates ZEB1 and breast cancer metastasis, without impacting primary tumor growth. Oncogene
35, 5977-5988 (2016).
33. Bhagwat, A. S. & Vakoc, C. R. Targeting Transcription Factors in Cancer. TRENDS in CANCER 1, 53-65 (2015).
34. De Val, S. et al. Combinatorial Regulation of Endothelial Gene Expression by Ets and Forkhead Transcription Factors. Cell 135, 1053-1064 (2008).
35. Yu, Y. et al. Panoramix enforces piRNA-dependent cotranscriptional silencing. Science 350, 339-342 (2015).
36. Rozhkov, N. V., et al. Multiple roles for Piwi in silencing Drosophila transposons. Genes Dev. 27, 400-412 (2013).
37. Paddison, P. J. et al. A resource for large-scale RNA-interference-based screens in mammals. Nature 428, 427-431 (2004).
38. Mehta, S. et al. Radiogenomics Monitoring in Breast Cancer Identifies Metabolism and Immune Checkpoints as Early Actionable Mechanisms of Resistance to Anti-angiogenic Treatment. EBioMedicine 10, 109-116 (2016).
39. Liang, W.-C. et al. Cross-species vascular endothelial growth factor (VEGF)-blocking antibodies completely inhibit the growth of human tumor xenografts and measure the contribution of stromal VEGF. J. Biol. Chem. 281, 951-961 (2006).
40. Roland, C. L., et al. Cytokine Levels Correlate with Immune Cell Infiltration after Anti-VEGF. (2009). doi:10.1371/journal.pone.0007669
41. Bruna, A. et al. A Biobank of Breast Cancer Explants with Preserved Intra-tumor Heterogeneity to Screen Anticancer Compounds. Cell 1-38 (2016). doi:10.1016/j.cell.2016.08.041
42. Han, X. et al. Tunicamycin enhances the antitumor activity of trastuzumab on breast cancer in vitro and in vivo. Oncotarget 6, 38912-38925 (2015).
43. Leslie, M. et al, June 2016, Tumors' do-it-yourself blood vessels. Science, 352 (6292):1381-1383

1. A method for increasing the sensitivity of a tumor to anti-angiogenic therapy comprising treating a subject having a tumor with an anti-angiogenic therapeutic composition or compound and substantially simultaneously inhibiting vascular, or vasculogenic, mimicry (VM).

2. The method according to claim 1, wherein inhibition of VM comprises further administering (a) a therapeutic compound that inhibits the activity or pathway of the transcription factor FOXC2; (b) a therapeutic compound that activates or enhances the activity or pathway of IRE1 or inhibits the activity of its target genes; or (c) administering both said therapeutic compounds (a) and (b).

3. The method according to claim 1, comprising treating the subject with an anti-angiogenic therapeutic composition or compound, a therapeutic compound that inhibits the activity or pathway of the transcription factor FOXC2; and a therapeutic compound that activates or enhances the activity or pathway of IRE1 or inhibits the activity of its target genes.

4. The method according to claim 2, comprising administering the compounds in a single composition or substantially simultaneously.

5. The method according to claim 2, comprising administering the compounds sequentially.

6. The method according to claim 1, wherein the tumor is a breast cancer tumor.

7. The method according to claim 1, wherein the anti-angiogenic therapeutic compound is an antagonist or antibody to VEGF, an antagonist or antibody to FGF, an antagonist, inhibitor or antibody to EGFR, an antagonist, inhibitor or antibody to PDGF, an antagonist, inhibitor or antibody to PDEGFR, an angiostatic steroid, a kinase inhibitor, thalidomide, itraconazole, carboxyamidotriazole, TNP-470, CM101, IFN-α, IL-12, platelet factor-4, suramin and its analogs, SU5416, thrombospondin, cartilage-derived angiogenesis inhibitory factor, matrix metalloproteinase inhibitor, angiostatin, endostatin, 2-methoxyestradiol, tecogalan, tetrathiomolybdate, prolactin, anti-integrin alpha v beta 3 antibody or inhibitor, linomide, or tasquinimod.

8. The method according to claim 2, wherein inhibiting the FOXC2 pathway comprises inhibiting one of more of the FOXC2 pathway targets: MEF2C, SERPINE2, SLPI, GREM1, TMEM100, SERPINE1, CYP1B1, ANGPTL4, FGF2, PRKCA, PRKD1, ITGA5, GATA6, DDAH1, ADM, HMOX1, HIPK2, CCBE1, IL8, WNT5A, PTK2B, ECM1, HIF1A, SRPX2, TBXA2R, HSPB1, SPHK1, HGF, RAPGEF2, C3AR1, HDAC9, C5AR1, PDGFB, MTDH, RRAS, RHOB, SIRT1, CIB1, CCL5, ERAP1, C190RF10, BTG1, PIK3R6, PLCG1, EGR1, ITGB2, GATA4, PHACTR1, RCAN2, SOBP, VCAN, FRY, FAM129A, GLIPR1, OSR1, NOV, EPS8, VIM, SDC2, COL6A2, WWTR1, TSC22D1, ENO2, ABI3BP, FOXL1, VASN, MYLK, PPP1R3C, DOCK10, KANK2, FN1, ANGPT1, LGALS3BP, CAMK1D, SOD3, CXXC5, CSGALNACT1, PNRC1, HTRA3, SDC3, SPP1, PLSCR4, ICAM1, TSPAN15, OSMR, KDELR3, TRIOBP, GBP4, ANGPTL2, TRIB2, SLC15A3.

9. The method according to claim 8, wherein the therapeutic compound that inhibits FOXC2 or the FOXC2 pathway is a p38 MAPK inhibitor, a Cdk/Cdk5 inhibitor, a PDGFR inhibitor, a PKA inhibitor, a PKD inhibitor, a PI3K inhibitor, a MET inhibitor, a CAMK inhibitor, a FGFR inhibitor, or a blocking antibody against the said targets or their ligands.

10. The method according to claim 2, wherein the IRE-1 pathway target gene is one or more of: MGP, RBP1, SLPI, SERPINE2, AQP1, SFRP1, ICAM1, ANK, COL6A1, PROS1, PLSCR4, HTRA3, DECR1, NEURL3, ZHX1, PFN2, DMP1, IL1R1, NOD1, PADI2, RBP2, GCHFR, SAMSN1, C1QTNF1, ABCG1, TFDP2, PAPLN, TNFRSF9, OAF, PLAT, TSLP, MEGF6, H2AFV, ADD2, PADI3, DUSP27, GSTT1, S100A4, DNAJC12, HSPB1, SCN5A, NOV, CTSH, PRKG2, NGEF, FSD1L, UGDH, FBLIM1, LIX1L, AKR1C13, LPXN, DUSP6, RNF130, PTGR1, TMOD2, CST3, ANKRD6, RTKN2, IL12RB1, LDHB, BENDS, GM10471, SPN, RAET1E, RIN2, PDE6D, GNB4, MCTP1, PER3, LHPP, CALR3, CADM1, ITGB2, GHR, CRIP1, MSRB2, EGR2, PAQR7 DOK1, ACSBG1, LEPROT, FAM131B, GPRIN3, COL16A1, GRAP, FKBP1B, GSTMS, KANK2, PSG17, PIK3CD, INF2, MYLK, EML1, TDRD7, ALDH7A1, FAM219A, SH3BGRL, FAM221A, FAM102B, FN1, MAGED2, NUSAP1, M1AP, CISH, TBC1D2B, ATPIF1, MGST3, CNP, XKRS, NEIL3, RALGPS2, MTCH1, CAND2, MEST, TMEM243, XRCC3, NINJ2, ECM1, CPNE3, RAF1, SEPN1, CHST12, NADSYN1, CX3CL1, CD82, CDHR1, PEAR1, POLD4, NR2F1, FHL2, ATHL1, CDKN2AIPNL, RAET1D, SCARA3, PLSCR2, CRTAP.

11. The method according to claim 10, wherein the therapeutic compound that activates the activity of IRE1 is thapsigagin, DTT, brefaldin A, bortezimib, acetaminophen, amiodarone, arsenic trioxide, Bleomycin, cisplatin, clozapine, olanzapine, cyclosporin, diclofenac, indomethacin, efavirenz, Proteasome inhibitors, zidovudine, sertraline, troglitazone, erlotinib, or doxorubicin.

12. The method according to claim 10, wherein the therapeutic compound inhibits said target of the IRE1 pathway.

13. The method according to claim 12, wherein the therapeutic compound is an antibody to an IRE1 pathway target.

14. The method according to claim 1, wherein said subject has breast cancer.

15. The method according to claim 1, wherein said subject's tumor is, or becomes over a period of time, refractory to treatment with anti-angiogenic therapy.

16. The method according to claim 1, comprising treating a mammalian subject having a tumor with an antibody to VEGF and substantially simultaneously inhibiting vascular, or vasculogenic, mimicry (VM).

17. The method according to claim 16, wherein inhibition of VM comprises further administering (a) a therapeutic compound that inhibits the activity or pathway of the transcription factor FOXC2; (b) a therapeutic compound that activates or enhances the activity or pathway of IRE1 or inhibits the activity of its target genes, or (c) both said therapeutic compounds (a) and (b).

18. A therapeutic composition for inhibiting tumor vascularization and vasculogenic mimicry comprising in a suitable pharmaceutical carrier, an anti-angiogenic therapeutic compound and at least one of (a) a therapeutic compound that inhibits the activity or pathway of the transcription factor FOXC2; and (b) a therapeutic compound that activates or enhances the activity or pathway of IRE1 or inhibits the activity of its targets.

19. The composition according to claim 18, comprising said anti-angiogenic therapeutic compound, said compound (a) and said compound (b).

20. A method for the treatment of cancer comprising treating a mammalian subject having a tumor with an anti-angiogenic therapeutic composition or compound and substantially simultaneously inhibiting vascular, or vasculogenic, mimicry (VM).
